(19) United States
(12) Patent Application Publication
(10) Pub. No.: US 2026/0009030 A1
(43) Pub. Date: Jan. 8, 2026

(54) COMPOUND FOR INHIBITING AGT GENE EXPRESSION, AND PHARMACEUTICAL COMPOSITION AND USE THEREOF

(71) Applicant: RIGERNA THERAPEUTICS (SUZHOU) CO., LTD., SUZHOU, JIANGSU (CN)

(72) Inventors: YUANYU HUANG, SUZHOU, JIANGSU (CN); HAITAO LI, SUZHOU, JIANGSU (CN); LINA KONG, SUZHOU, JIANGSU (CN); XIAOFENG HAN, SUZHOU, JIANGSU (CN)

(21) Appl. No.: 19/127,659

(22) PCT Filed: Apr. 9, 2024
(86) PCT No.: PCT/CN2024/086788

§ 371 Date: May 6, 2025

(30) Foreign Application Priority Data
Apr. 14, 2023 (WO) PCT/CN2023/088483
Aug. 30, 2023 (CN) 202311107991.9

(51) Int. Cl. C12N 15/113; A61P 9/12

(52) U.S. Cl. CPC C12N 15/113; A61P 9/12; C12N 2310/11; C12N 2310/14; C12N 2310/315; C12N 2310/321; C12N 2310/322; C12N 2310/351

(57) ABSTRACT

Provided are a compound for inhibiting angiotensinogen (AGT) gene expression, and a pharmaceutical composition and the use thereof, which belong to the technical field of small nucleic acid drug delivery. The provided oligonucleotide-conjugated compound and a pharmaceutical composition thereof can induce silencing complex (RISC)-mediated cleavage of RNA transcripts of the AGT gene, thereby inhibiting the expression of the AGT gene. The provided oligonucleotide-conjugated compound and the pharmaceutical composition thereof help to alleviate, prevent and/or treat diseases or conditions mediated by the AGT gene.

### FIELD OF THE DISCLOSURE

The present disclosure relates to the technical field of small nucleic acid drug delivery, and particularly relates to an oligonucleotide-conjugated compound for inhibiting AGT gene expression, and pharmaceutical composition and use thereof.

### BACKGROUND OF THE DISCLOSURE

Angiotensinogen (AGT), also known as SERPINA8 or ANHU, is a member of the serpin family and a component of the renin-angiotensin-aldosterone system (RAAS). RAAS plays a crucial role in blood pressure regulation. Juxtaglomerular cells of the kidney secrete renin into the circulation. AGT is mainly produced in the liver and released into the circulation, where renin converts AGT into angiotensin I. Subsequently, angiotensin I was converted into angiotensin II through angiotensin-converting enzyme (ACE). Angiotensin II is a peptide hormone that causes vasoconstriction, which in turn can increase blood pressure. Angiotensin II also stimulates the secretion of the hormone aldosterone of the adrenal cortex. Aldosterone causes the kidney to increase the reabsorption of sodium and water, leading to an increase in the body's fluid volume, which in turn can increase blood pressure. Excessive stimulation or activity of the RAAS pathway can lead to high blood pressure. Chronic high blood pressure is called hypertension. In patients with hypertension, the high blood pressure requires the heart to work harder to circulate blood through the blood vessels.

The World Health Organization (WHO) has identified hypertension as a major cause of cardiovascular morbidity. Hypertension is a major risk factor for various diseases, disorders and conditions, such as shortened life expectancy, chronic kidney diseases, stroke, myocardial infarction, heart failure, vascular aneurysms (e.g., aortic aneurysm), peripheral arterial diseases, heart damage (e.g., cardiac dilation or hypertrophy) and other cardiovascular-related diseases, disorders, and/or conditions.

Although there is a large number of antihypertensive drugs available for treating hypertension, more than two-thirds of subjects cannot have their blood pressure controlled with a single antihypertensive drug and need two or more antihypertensive drugs selected from different drug classes. Adherence and side-effects increase with the increase in the number of medications used, which further reduces the number of subjects with controlled blood pressure.

Therefore, providing an oligonucleotide-conjugated compound for inhibiting the expression of AGT gene is of great significance for improving the disease course of hypertensive patients, alleviating, preventing, and/or treating a disease or disorder mediated by AGT gene.

### SUMMARY OF THE DISCLOSURE

In the first aspect of the present disclosure, the present disclosure provides an oligonucleotide-conjugated compound having a structure represented by Formula (I) or a pharmaceutically acceptable salt thereof:

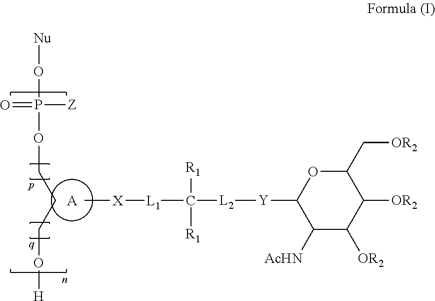

wherein, in the structure of the Formula mentioned above:
each A is independently selected from unsubstituted or substituted 4-10-membered aliphatic rings;
n is selected from 1, 2, 3 or 4;
each Z is independently hydroxyl or sulfydryl;
each p is independently selected from 1, 2 or 3;
each q is independently selected from 1, 2 or 3;
each X is independently selected from NH, O or S;
each L₁ is independently selected from

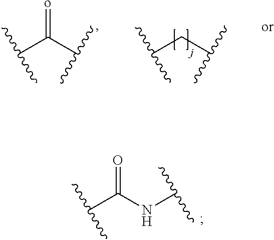

wherein, j is selected from 1, 2, 3, 4, 5, 6, 7, 8, 9 or 10;
each R₁ is independently selected from H, C₁-C₆ alkyl, C₁-C₆ halogenated alkyl or C₁-C₆ alkoxy;
each L₂ is independently selected from C₁-C₃₀ alkylene or

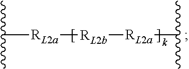

wherein, each R_{L2a} is independently selected from C₁-C₁₀ alkylene, each R_{L2b} is independently selected from O, S, NH or —NH—C(O)—, k is selected from 1, 2, 3, 4, 5, 6, 7, 8, 9 or 10;
each Y is independently selected from NH, O or S;
each R₂ is independently selected from: H,

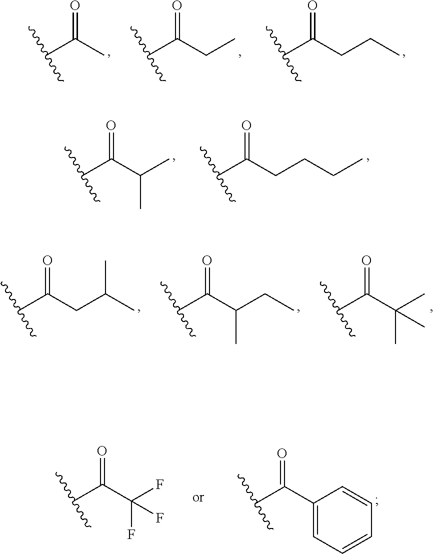

wherein, Nu represents a double-stranded oligonucleotide or a pharmaceutically acceptable salts thereof used for inhibiting the expression of AGT gene in a cell; the double-stranded oligonucleotide comprises a sense strand and an antisense strand, the sense strand and the antisense strand form a duplex region, and the antisense strand comprises a complementary sequence that is complementary to the AGT mRNA target sequence, the target sequence is selected from a nucleotide region consisting of 14-35 consecutive nucleotides on AGT mRNA.

In the double-stranded oligonucleotide represented by Nu, the sense strand comprises a nucleotide sequence of at least 17, at least 18, or at least 19 consecutive nucleotides in any one of the sequences of SEQ ID NO:1, 3, 5, 7, 9, or 11, or comprises a nucleotide sequence having 1, 2, or 3 nucleotide differences from the consecutive nucleotides; and/or, the antisense strand comprises a nucleotide sequence of at least 17, at least 18, or at least 19 consecutive nucleotides in any one of the sequences of SEQ ID NO:2, 4, 6, 8, 10, or 12, or comprises a nucleotide sequence having 1, 2, or 3 nucleotide differences from the consecutive nucleotides.

In some specific embodiments of the present disclosure, in the double-stranded oligonucleotide represented by Nu, in the direction of 5′-3′, the antisense strand comprises the 1st-19th consecutive nucleotides in any one of the sequences of SEQ ID NO: 2, 4, 6, 8, 10, or 12, or comprises a nucleotide sequence having 1, 2, or 3 nucleotide differences from the consecutive nucleotides.

In some specific embodiments of the present disclosure, the sense strand of the double-stranded oligonucleotide represented by Nu comprises any one of the sequences shown in SEQ ID NO. 1, 3, 5, 7, 9, or 11, or a nucleotide sequence having 1 or 2 nucleotide differences from any of the above sequences; and/or, the antisense strand of the double-stranded oligonucleotide comprises any one of the sequences shown in SEQ ID NO. 2, 4, 6, 8, 10, or 12, or a nucleotide sequence having 1 or 2 nucleotide differences from any of the above sequences.

In some specific embodiments of the present disclosure, each nucleotide in the double-stranded oligonucleotide is independently selected from the following modified nucleotides:
2′-fluoro-modified nucleotides, 2′-deoxy-modified nucleotides, 2′-O-methyl-modified nucleotides, 2′-O—(CH₂)ₓ—O—Rₘ-modified nucleotides, 2′-O—Si(Rₙ)₃-modified nucleotides, 2′-amino-modified nucleotides, abasic nucleotides or nucleotide analogs; the nucleotide analogs are selected from one or more of PNA, MNA, BNA, LNA, GNA, TNA or UNA; wherein, x is selected from 1 or 2, Rₘ is selected from optionally substituted C₁₋₆ alkyl or optionally substituted C₁₋₆ alkoxy, if Rₘ comprises a substituent, the substituent is selected from halogen, C₁₋₆ alkoxy, hydroxyl or amino, Rₙ is independently selected from optionally substituted C₁₋₆ alkyl.

In some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from nucleotides other than 2′-fluoro-modified nucleotides; at least four nucleotides among the nucleotides at positions 2nd, 6th, 9th-12th, 14th and 16th of the nucleotide sequence in the antisense strand are selected from 2′-fluoro-modified nucleotides, and the nucleotides at the remaining positions are independently selected from nucleotides other than 2′-fluoro-modified nucleotides.

In some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from 2′-O-methyl-modified nucleotides or 2′-O-methoxyethyl-modified nucleotides; at least four nucleotides among the nucleotides at positions 2nd, 6th, 9th-12th, 14th and 16th of the nucleotide sequence in the antisense strand are selected from 2′-fluoro-modified nucleotides, and the nucleotides at the remaining positions are selected from 2′-O-methyl-modified nucleotides or 2′-O-methoxyethyl-modified nucleotides.

In some specific embodiments of the present disclosure, in the direction from the 5′-end to the 3′-end, at least one of the linkages between the following nucleotides of the sense strand is a phosphorothioate linkage: the linkage between the first and the second nucleotides at the 5′-end of the sense strand; the linkage between the second and the third nucleotides at the 5′-end of the sense strand.

In some specific embodiments of the present disclosure, in the direction from the 5′-end to the 3′-end, at least one of the linkages between the following nucleotides of the antisense strand is a phosphorothioate linkage: the linkage between the first and the second nucleotides at the 5′-end of the antisense strand; the linkage between the second and the third nucleotides at the 5′-end of the antisense strand; the linkage between the first and the second nucleotides at the 3′-end of the antisense strand; the linkage between the second and the third nucleotides at the 3′-end of the antisense strand.

In some specific embodiments of the present disclosure, each nucleotide in the double-stranded oligonucleotide is selected from modified nucleotides;
the double-stranded oligonucleotide is selected from at least one of the following groups:

| | sense strand (5′-3′) | antisense strand (5′-3′) |
|---|---|---|
| group | CmsAmsAmGmUmUmGf | AmsAfsUmUmUmUfUmGmUmUfC |
| 1 | AfGfAfAmCmAmAmAm | mUmCmAfA(moe)CfUmUmGmsAm |
| | AmAmUmUm | sAm |
| group | GmsUmsUmUmUmAmAf | UmsAfsUmAmCmUfUmUmAmAm |
| 2 | AfAfUfUmAmAmAmGm | UfUmUmUfA(moe)AfAmAmCmsC |
| | UmAmUmAm | msCm |
| group | AmsAmsGmUmAmUmAf | AmsAfsUmGmCmAfAmAmAmAfU |
| 3 | CfAfUfUmUmUmUmGmC | mGmUmAfUmAfCmUmUmsUmsA |
| | mAmUmUm | m |
| group | AmsAmsGmUmAmUmAf | AmsAfsUmGmCmAfAmAmAmAfU |
| 4 | CfAfUfUmUmUmUmGmC | mGmUmAfT(moe)AfCmUmUmsUm |
| | mAmUmUm | sAm |

In some specific embodiments of the present disclosure, the oligonucleotide-conjugated compound is selected from any of the compounds shown in Table 6;
In some specific embodiments of the present disclosure, the compound is selected from any one of RZ003062, RZ003064, RZ003065, RZ003066, or RZ003089:

| | sense strand (5′-3′) | antisense strand (5′-3′) |
|---|---|---|
| RZ003062 | CmsAmsAmGmUmUmGfAf | AmsAfsUmUmUmUfUmGmUm |
| | GfAfAmCmAmAmAmAmA | UfCmUmCmAfA(moe)CfUmUm |
| | mUmUm_(CR01008 × 3) | GmsAmsAm |
| RZ003064 | GmsUmsUmUmUmAmAfAf | UmsAfsUmAmCmUfUmUmAm |
| | AfUfUmAmAmAmGmUmA | AmUfUmUmUfA(moe)AfAmA |
| | mUmAm_(CR01008 × 3) | mCmsCmsCm |
| RZ003065 | AmsAmsGmUmAmUmAfCf | AmsAfsUmGmCmAfAmAmAm |
| | AfUfUmUmUmUmGmCmA | AfUmGmUmAfUmAfCmUmUm |
| | mUmUm_(CR01008 × 3) | sUmsAm |
| RZ003066 | AmsAmsGmUmAmUmAfCf | AmsAfsUmGmCmAfAmAmAm |
| | AfUfUmUmUmUmGmCmA | AfUmGmUmAfT(moe)AfCmUm |
| | mUmUm_(CR01008 × 3) | UmsUmsAm |
| RZ003089 | UmsCmsAmAmCmUmGfGf | AmsGfsUmUmUmCfUmUmCfA |
| | AfUfGmAmAmGmAmAmA | mUmCmCmAfG(moe)UfUmGm |
| | mCmUm_(CR01008 × 3) | AmsGmsGm |

In the second aspect of the present disclosure, the present disclosure provides a pharmaceutical composition, the pharmaceutical composition comprises the oligonucleotide-conjugated compound according to the first aspect of the present disclosure.

In the third aspect of the present disclosure, the present disclosure provides a use of the oligonucleotide-conjugated compound according to the first aspect of the present disclosure or the pharmaceutical composition according to the second aspect of the present disclosure in the manufacture of a medicament for alleviating, preventing and/or treating a disease or disorder mediated by AGT gene.

In the fourth aspect of the present disclosure, the present disclosure provides a kit, the kit comprises the oligonucleotide-conjugated compound according to the first aspect of the present disclosure or the pharmaceutical composition according to the second aspect of the present disclosure.

In the fifth aspect of the present disclosure, the present disclosure provides a method for inhibiting the expression of AGT gene, which comprises administering to a subject the oligonucleotide-conjugated compound according to the first aspect of the present disclosure or the pharmaceutical composition according to the second aspect of the present disclosure.

In the sixth aspect of the present disclosure, the present disclosure provides a method for alleviating, preventing and/or treating a disease or disorder mediated by AGT, which comprises administering to a subject the oligonucleotide-conjugated compound according to the first aspect of the present disclosure or the pharmaceutical composition according to the second aspect of the present disclosure.

In some specific embodiments of the present disclosure, the disease or disorder mediated by AGT includes a disease associated with the mRNA level of the AGT gene expression.

In some specific embodiments of the present disclosure, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

The oligonucleotide-conjugated compound and pharmaceutical composition thereof provided by the present disclosure can induce silencing complex (RISC)-mediated cleavage of RNA transcripts of the AGT gene, the expression of AGT gene. The oligonucleotide-conjugated compound and pharmaceutical composition thereof provided by the present disclosure help to alleviate, prevent, and/or treat a disease or disorder mediated by angiotensinogen (AGT) gene.

### BRIEF DESCRIPTION OF THE DRAWINGS

FIG. 1: Changes of systolic blood pressure levels in hREN×hAGT hypertensive mice after administrating the oligonucleotide-conjugated compound in Example 2.

FIG. 2: Changes of AGT protein levels in hREN×hAGT hypertensive mice after administrating the oligonucleotide-conjugated compound in Example 3.

### DETAILED DESCRIPTION OF THE EXEMPLARY EMBODIMENTS

The present disclosure discloses an oligonucleotide-conjugated compound, a pharmaceutical composition, and use thereof that inhibits the expression of AGT gene. Those skilled in the art can draw on the content herein and appropriately modify the process parameters to achieve this. It should be particularly noted that all similar substitutions and modifications are obvious to those skilled in the art, and they are all considered to be included in the present disclosure. The methods and applications of the present disclosure have been described through preferred examples. It is obvious to relevant personnel that they can make changes or appropriate alterations and combinations to the methods and applications herein without departing from the content, spirit, and scope of the present disclosure, so as to implement and apply the technology of the present disclosure.

### Explanation of Terms

In the present disclosure, the terms “comprise” or “include” are open-ended expressions, meaning that they include the content specified in the present disclosure, but do not exclude other aspects.

In the present disclosure, the terms “optionally,” “optional,” or “option” generally mean that the subsequent event or condition may or may not occur, and the description includes the case where the event or situation occurs and the case where the event or situation does not occur.

In the present disclosure, the term “small interfering RNA (siRNA)” means a double-stranded RNA that is 17 to 25 nucleotides in length and consists of a sense strand and an antisense strand. siRNA mediates the targeted cleavage of RNA transcripts in the RISC pathway through forming an RNA-induced silencing complex (RISC). Specifically, siRNA directs the specific degradation of mRNA sequences through the well-known RNA interference (RNAi) process, inhibiting the translation of mRNA into amino acids and the conversion into proteins. For example, siRNA can regulate (e.g., inhibit) the expression of AGT in a cell.

In the present disclosure, the term “antisense strand (or referred to guide strand)” comprises a region that is substantially complementary to a target sequence, such as the mRNA of AGT. The “sense strand (or referred to passenger strand)” means the strand that comprises an iRNA strand that is substantially complementary to the antisense strand. The term “substantially complementary” means completely complementary or at least partially complementary. For example, the antisense strand is completely complementary or at least partially complementary to the target sequence. In the case of partially complementary, mismatches can exist within the internal or terminal regions of the molecule. Wherein, the most tolerated mismatches are present within the terminal regions, for example, within 5, 4, 3, or 2 nucleotides at the 5′-end and/or 3′-end of the iRNA.

It should be noted that the “at least partially substantially complementary” between the antisense strand and mRNA means that the antisense strand has a polynucleotide that is substantially complementary to a continuous part of the mRNA of interest (e.g., mRNA encoding AGT). Alternatively, if a polynucleotide is substantially non-discontinuously complementary to a part of the mRNA encoding AGT, then the antisense strand is complementary to at least a part of the mRNA of AGT.

In the present disclosure, the term “target sequence” means a continuous portion of the nucleotide sequence of an mRNA molecule formed during the transcription of the encoding the AGT gene, including the mRNA that is a product of RNA processing of the primary transcript. The AGT can be present within cells, for example, cells within a subject.

In the present disclosure, the term “complementary” means the ability of an oligonucleotide of the first sequence to hybridize with an oligonucleotide of the second sequence under certain conditions and form a double-stranded structure.

In the present disclosure, the term “substantially complementary” means that in the duplex region, the sense strand and the antisense strand have no more than 3 nucleotide mismatches, no more than 2 nucleotide mismatches, or no more than 1 nucleotide mismatch, such as 3 nucleotide mismatches, 2 nucleotide mismatches, 1 nucleotide mismatch, or 0 nucleotide mismatches, at the same time, retaining the ability to hybridize under relevant conditions. Additionally, when two oligonucleotides are designed to form one or more single-stranded overhangs upon hybridization, such overhangs should not be regarded as mismatches in terms of complementarity. In the present disclosure, when meeting the requirements of the hybridization ability above, “complementary” sequences can also include or be entirely formed from non-Watson-Crick base pairs and/or base pairs formed from non-natural and modified nucleotides. Such non-Watson-Crick base pairs include, but are not limited to, G:U wobble base pairing or Hoogstein base pairing. Correspondingly, in the present disclosure, unless otherwise specified, a “mismatch” means in the siRNA duplex molecules, the bases at corresponding positions do not pair in a complementary manner.

In the present disclosure, the term “complementary sequence with complementarity” means that in the complementary sequence between the antisense strand of double-stranded oligonucleotide and AGT mRNA, the number of mismatches does not exceed 3 nucleotides, does not exceed 2 nucleotides, or does not exceed 1 nucleotide. For example, 3 nucleotide mismatches, 2 nucleotide mismatches, 1 nucleotide mismatch, or 0 nucleotide mismatches.

In the present disclosure, the terms “nucleotide difference” and “nucleoside base difference”, and the term “difference of nucleotide sequence” can be used interchangeably. They means that compared to the original nucleotide sequence, the base type of a nucleotide at the same or corresponding position has changed. For example, when a nucleoside base in the original nucleotide sequence is A, and at the same or corresponding position, the nucleoside base is changed to U, C, G, or dT, dC, dG, etc., a difference in the nucleotide sequence is considered to exist at the position. It should be noted here that, when compared to the original nucleotide sequence, the nucleotide at the same or corresponding position only differs in terms of the presence or type of modification, a difference in the nucleotide sequence is not considered to exist at the position.

In the present disclosure, the term “overhang” means at least one unpaired nucleotide protruding from the double-helix structure of the double-stranded oligonucleotide, and it is also the nucleotide sequence in the siRNA structure other than the double-stranded region. For example, when the 3′-end of one strand of the sense strand and/or the antisense strand extends beyond the 5′-end of the other strand, or when the 5′-end of one strand of the sense strand and/or the antisense strand extends beyond the 3′-end of the other strand, there is a nucleotide overhang. The overhang can comprise at least one nucleotide, at least two nucleotides, at least three nucleotides, at least four nucleotides, at least five nucleotides or more nucleotides. The nucleotide overhang can comprise or be composed of nucleotide/nucleoside analogs, including deoxynucleotides/nucleosides. The overhang can be located on the sense strand, the antisense strand, or any combination thereof. In addition, the overhang nucleotides can appear at the 5′-end, 3′-end of the antisense or sense strand, or at both ends.

In the present disclosure, the term “DEPC H₂O” means ultrapure water (grade 1 water) that has been treated with diethyl pyrocarbonate (DEPC) and sterilized under high temperature and high pressure.

In the present disclosure, the term “subject” means any animals that are examined, studied, or treated. The present disclosure is not intended to be limited by any specific type of subject. In some embodiments of the present disclosure, human is the preferred subject. In other embodiments, non-human animals are the preferred subjects, including but not limited to mice, monkeys, ferrets, cattle, sheep, goats, pigs, chickens, turkeys, dogs, cats, horses, and reptiles.

In the present disclosure, the term “inhibiting the expression of AGT gene” includes inhibition of the AGT gene at any level. For example, at least partial inhibition of the AGT gene expression, such as inhibiting at least approximately 20%, at least approximately 25%, at least approximately 30%, at least approximately 35%, at least approximately 40%, at least approximately 45%, at least approximately 50%, at least approximately 55%, at least approximately 60%, at least approximately 65%, at least approximately 70%, at least approximately 75%, at least approximately 80%, at least approximately 85%, at least approximately 90%, at least approximately 91%, at least approximately 92%, at least approximately 93%, at least approximately 94%, at least approximately 95%, at least approximately 96%, at least approximately 97%, at least approximately 98%, or at least approximately 99%. Wherein, the AGT gene expression can be evaluated based on the level of any variable related to the AGT gene expression, such as the mRNA level or protein level of AGT. The inhibition can be evaluated by a decrease of the absolute or relative level of one or more of these variables compared to a control level. The control level can be any type of control level utilized in the art, for example, the baseline level before administration, or the level measured in similar subjects, cells, or samples that have not been treated or have been treated with a control (such as a buffer-only control or an inactive agent control).

In the present disclosure, “conjugation” means the connection of two or more chemical moieties to each other through covalent bonding. The “conjugate” means a compound formed by the covalent bonding of various chemical moieties. The “conjugated molecule” is understood to be a specific compound that can be conjugated to an oligonucleotide through a reaction to ultimately form the oligonucleotide conjugate of the present disclosure.

In the present disclosure, the “pharmaceutical composition” can be used for treating diseases as well as for in-vitro cell culture experiments. When used for treating diseases, the term “pharmaceutical composition” generally means a unit-dose form and can be prepared by any of the methods well-known in the pharmaceutical field. All methods include the step of combining the active ingredient with excipients that constitute one or more accessory ingredients. Usually, the composition is prepared by uniformly and thoroughly combining the active siRNA with liquid excipients, finely divided solid excipients, or both.

In the present disclosure, the term “pharmaceutically acceptable” means a substance or composition must be chemically and/or toxicologically compatible with the other ingredients of the formulation and/or with the mammal being treated therewith. Preferably, the “pharmaceutically acceptable” of the present disclosure means approved by federal regulatory agencies or national governments, or are listed in the United States Pharmacopeia or other generally recognized pharmacopeias for use in animals, especially in human.

In the present disclosure, the term “pharmaceutically acceptable excipient” each can include any solvents, solid excipients, diluents, or other liquid excipients, etc., that are suitable for the specific target dosage form. Their use is also within the scope considered by the present disclosure, except to the extent that any conventional excipient is incompatible with the siRNA of the present disclosure, such as any adverse biological effects produced or any interactions with any other components of the pharmaceutically acceptable composition in a harmful manner.

In the present disclosure, the term “treatment” means the use to obtain a desired pharmacological and/or physiological effect. The effect can be prophylactic in terms of completely or partially preventing diseases or symptoms thereof, and/or can be therapeutic in terms of partially or completely curing diseases and/or the adverse effects caused by the diseases. “Treatment” as used herein encompasses diseases in mammals, particularly human, and includes: (a) preventing the occurrence of a disease or disorder in an individual who is susceptible to the disease but has not yet been diagnosed with it; (b) inhibiting the disease, such as arresting its progression; or (c) alleviating the disease, such as reducing the symptoms associated with the disease. “Treatment” as used herein covers any administration of a drug or siRNA to an individual to treat, cure, relieve, improve, reduce, or inhibit the disease of the individual, including but not limited to administering a drug comprising the siRNA or siRNA conjugate described herein to an individual in need.

Except for any conventional excipients, within the scope of not being incompatible with the siRNA of the present disclosure, such as any adverse biological effects produced or any interactions with any other components of the pharmaceutically acceptable composition in a harmful way, their uses are also within the scope considered by the present disclosure.

### Oligonucleotide-Conjugated Compounds

In the first aspect of the present disclosure, the present disclosure provides an oligonucleotide-conjugated compound, which has a structure represented by Formula (I) or a pharmaceutically acceptable salt thereof:

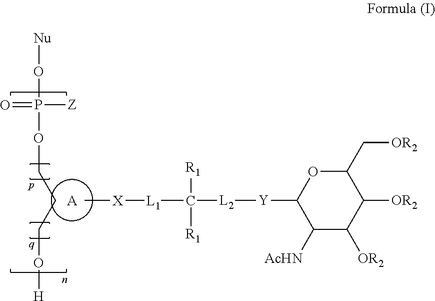

wherein, in the structure of the Formula mentioned above:
each A is independently selected from unsubstituted or substituted 4-10-membered aliphatic rings.
n is selected from 1, 2, 3 or 4.
Each Z is independently hydroxyl or sulfydryl.
Each p is independently selected from 1, 2 or 3.
Each q is independently selected from 1, 2 or 3.
Each X is independently selected from NH, O or S.
Each L₁ is independently selected from

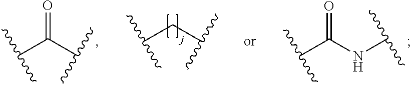

wherein, j is selected from 1, 2, 3, 4, 5, 6, 7, 8, 9 or 10.
Each R₁ is independently selected from H, C₁-C₆ alkyl, C₁-C₆ halogenated alkyl or C₁-C₆ alkoxy.
Each L₂ is independently selected from C₁-C₃₀ alkylene or

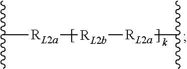

wherein, each R_{L2a} is independently selected from C₁-C₁₀ alkylene, each R_{L2b} is independently selected from O, S, NH or —NH—C(O)—, k is selected from 1, 2, 3, 4, 5, 6, 7, 8, 9 or 10.
Each Y is independently selected from NH, O or S.
Each R₂ is independently selected from: H,

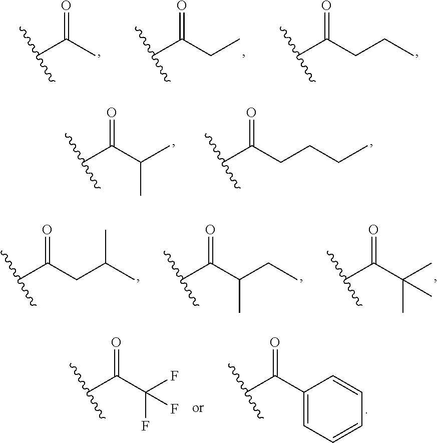

In some embodiments of the present disclosure, each A is independently substituted or unsubstituted 4-10-membered cycloalkyl or substituted or unsubstituted 4-10-membered cycloalkenyl group.

In some embodiments of the present disclosure, each A is independently substituted or unsubstituted 4-10-membered cycloalkyl.

In some embodiments of the present disclosure, each A is independently 4-10-membered cycloalkyl, for example monocyclic, spirocyclic or bridged cyclic.

In some embodiments of the present disclosure, each A is independently

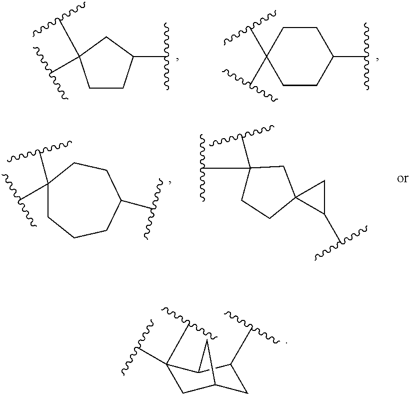 [SMILES: *C1CCC(*)(*)C1 ; *C1CCC(*)(*)CC1 ; *C1CCCC(*)(*)CC1 ; *C1CC12CCC(*)(*)C2 ; *C1CC2CC1C(*)(*)C2]

In some specific embodiments of the present disclosure, each A is

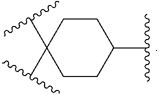 [SMILES: *C1CCC(*)(*)CC1]

In some specific embodiments of the present disclosure, the oligonucleotide-conjugated compound has a structure represented by Formula (II) or pharmaceutically acceptable salt thereof:

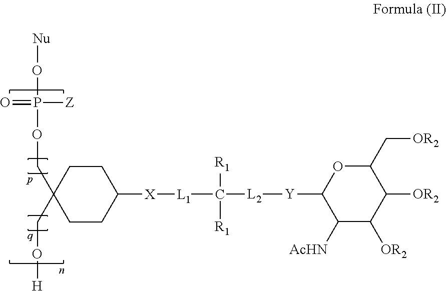

in Formula (II), p, q, n, Z, X, Y, L₁, L₂ and the substituent R₁ are as defined above, R₂ is selected from H.

In some specific embodiments of the present disclosure, each X is NH.

In some specific embodiments of the present disclosure, each L₁ is

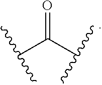 [SMILES: *C(*)=O]

In some specific embodiments of the present disclosure, the oligonucleotide-conjugated compound has a structure represented by Formula (III) or pharmaceutically acceptable salt thereof:

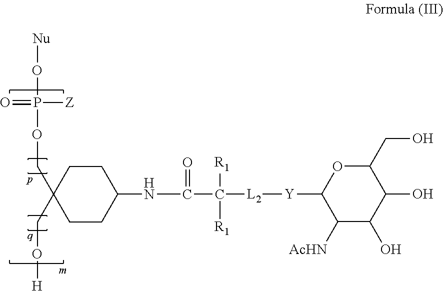

in Formula (III), m is selected from 1, 2, 3 or 4; the remaining substituents are as defined above.

In some specific embodiments of the present disclosure, each Z is hydroxyl.

In some embodiments of the present disclosure, each p is independently 1 or 2.

In some specific embodiments of the present disclosure, each p is 1.

In some embodiments of the present disclosure, each q is independently 1 or 2.

In some specific embodiments of the present disclosure, each q is 1.

In some specific embodiments of the present disclosure, each p is 1, and each q is 1.

In some specific embodiments of the present disclosure, each R₁ is H.

In some specific embodiments of the present disclosure, each Y is O.

Further, in some specific embodiments of the present disclosure, the oligonucleotide-conjugated compound has a structure represented by Formula (IV) or a pharmaceutically acceptable salt thereof:

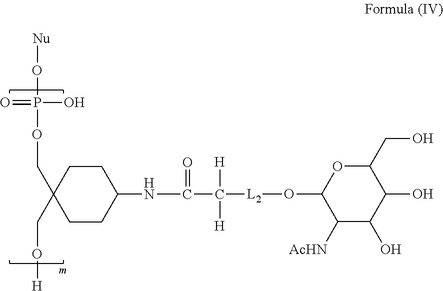

in Formula (IV), m is selected from 1, 2, 3 or 4; L₂ is independently selected from

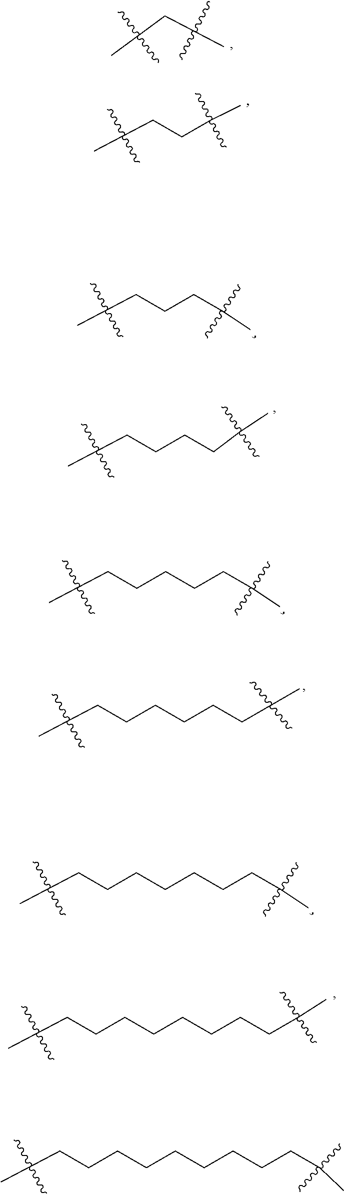

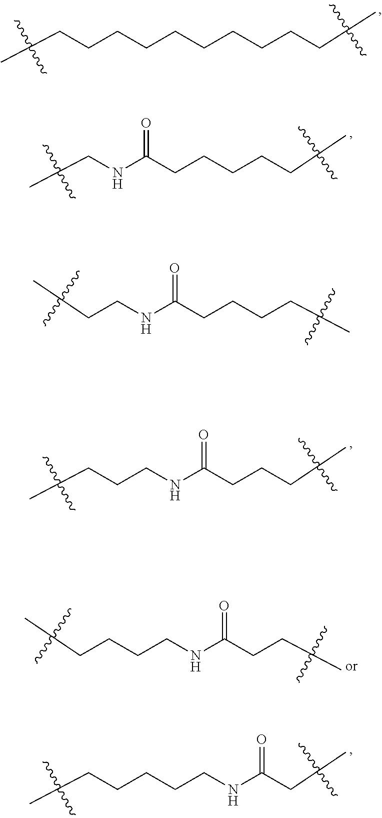 [SMILES: *CCCCCCCCCC* ; *CCCCCC(=O)NC* ; *CCCCC(=O)NCC* ; *CCCNC(=O)CCC* ; *CCCCNC(=O)CC* ; *CCCCCNC(=O)C*]

In some optional embodiments of the present disclosure, each L₂ is independently selected from

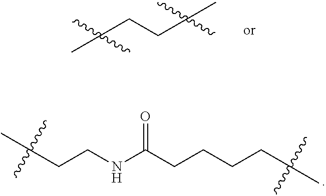

In some specific embodiments of the present disclosure, L₂ is selected from

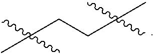 [SMILES: *CC*]

In some specific embodiments of the present disclosure, L₂ is selected from

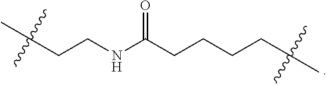 [SMILES: *CCCCC(=O)NCC*]

In some specific embodiments of the present disclosure, the oligonucleotide-conjugated compound has a structure represented by the following Formula or pharmaceutically acceptable salt thereof:

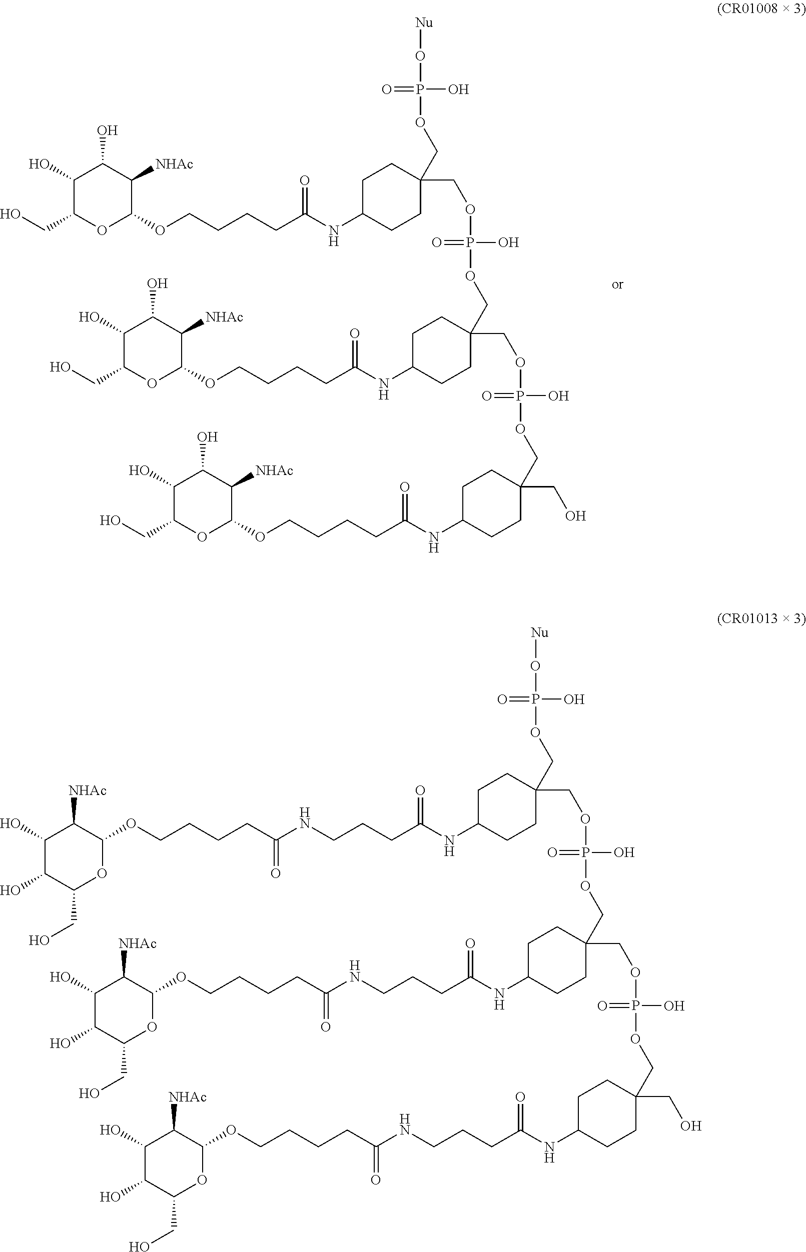

wherein, Nu represents a double-stranded oligonucleotide or a pharmaceutically acceptable salt thereof used for inhibiting the expression of AGT gene. The double-stranded oligonucleotide comprises a sense strand and an antisense strand. Wherein, the sense strand and the antisense strand are complementary or substantially complementary to form a double-stranded region. The substantially complementary means that the sense strand and the antisense strand have no more than 3 nucleotide mismatches in the double-stranded region.

And the antisense strand comprises a complementary sequence that is complementary to the AGT mRNA target sequence. The target sequence is selected from the nucleotide region on the AGT mRNA consisting of 14-35 consecutive nucleotides. Further, in the direction from the 5′-end to the 3′-end, the nucleotides at positions 2-19 of the antisense strand comprise a complementary region that is complementary to the AGT mRNA sequence.

In some specific embodiments of the present disclosure, the gene sequence of AGT is shown in SEQ ID No. 19:

| (SEQ ID No. 19) | |
|---|---|
| 1 | gaagaagctg ccgttgttct gggtactaca gcagaagggt atgcggaagc gagcacccca |
| 61 | gtctgagatg gctcctgccg gtgtgagcct gagggccacc atcctctgcc tcctggcctg |
| 121 | ggctggcctg gctgcaggtg accgggtgta catacacccc ttccacctcg tcatccacaa |
| 181 | tgagagtacc tgtgagcagc tggcaaaggc caatgccggg aagcccaaag accccacctt |
| 241 | catacctgct ccaattcagg ccaagacatc ccctgtggat gaaaaggccc tacaggacca |
| 301 | gctggtgcta gtcgctgcaa aacttgacac cgaagacaag ttgagggccg caatggtcgg |
| 361 | gatgctggcc aacttcttgg gcttccgtat atatggcatg cacagtgagc tatggggcgt |
| 421 | ggtccatggg gccaccgtcc tctccccaac ggctgtcttt ggcaccctgg cctctctcta |
| 481 | tctgggagcc ttggaccaca cagctgacag gctacaggca atcctgggtg ttccttggaa |
| 541 | ggacaagaac tgcacctccc ggctggatgc gcacaaggtc ctgtctgccc tgcaggctgt |
| 601 | acagggcctg ctagtggccc agggcagggc tgatagccag gcccagctgc tgctgtccac |
| 661 | ggtggtgggc gtgttcacag ccccaggcct gcacctgaag cagccgtttg tgcagggcct |
| 721 | ggctctctat acccctgtgg tcctcccacg ctctctggac ttcacagaac tggatgttgc |
| 781 | tgctgagaag attgacaggt tcatgcaggc tgtgacagga tggaagactg gctgctccct |
| 841 | gatgggagcc agtgtggaca gcaccctggc tttcaacacc tacgtccact tccaagggaa |
| 901 | gatgaagggc ttctccctgc tggccgagcc ccaggagttc tgggtggaca acagcacctc |
| 961 | agtgtctgtt cccatgctct ctggcatggg caccttccag cactggagtg acatccagga |
| 1021 | caacttctcg gtgactcaag tgcccttcac tgagagcgcc tgcctgctgc tgatccagcc |
| 1081 | tcactatgcc tctgacctgg acaaggtgga gggtctcact ttccagcaaa actccctcaa |
| 1141 | ctggatgaag aaactatctc cccggaccat ccacctgacc atgccccaac tggtgctgca |
| 1201 | aggatcttat gacctgcagg acctgctcgc ccaggctgag ctgcccgcca ttctgcacac |
| 1261 | cgagctgaac ctgcaaaaat tgagcaatga ccgcatcagg gtgggggagg tgctgaacag |
| 1321 | catttttttt gagcttgaag cggatgagag agagcccaca gagtctaccc aacagcttaa |
| 1381 | caagcctgag gtcttggagg tgaccctgaa ccgcccattc ctgtttgctg tgtatgatca |
| 1441 | aagcgccact gccctgcact tcctgggccg cgtggccaac ccgctgagca cagcatgagg |
| 1501 | ccagggcccc agaacacagt gcctggcaag gcctctgccc ctggcctttg aggcaaaggc |
| 1561 | cagcagcaga taacaacccc ggacaaatca gcgatgtgtc acccccagtc tcccaccttt |
| 1621 | tcttctaatg agtcgacttt gagctggaaa gcagccgttt ctccttggtc taagtgtgct |
| 1681 | gcatggagtg agcagtagaa gcctgcagcg gcacaaatgc acctcccagt ttgctgggtt |
| 1741 | tattttagag aatgggggtg gggaggcaag aaccagtgtt tagcgcggga ctactgttcc |
| 1801 | aaaaagaatt ccaaccgacc agcttgtttg tgaaacaaaa aagtgttccc ttttcaagtt |
| 1861 | gagaacaaaa attgggtttt aaaattaaag tatacatttt tgcattgcct tcggtttgta |
| 1921 | tttagtgtct tgaatgtaag aacatgacct ccgtgtagtg tctgtaatac cttagttttt |
| 1981 | tccacagatg cttgtgattt ttgaacaata cgtgaaagat gcaagcacct gaatttctgt |
| 2041 | ttgaatgcgg aaccatagct ggttatttct cccttgtgtt agtaataaac gtcttgccac |
| 2101 | aataagcctc caaaaa |

In some specific embodiments of the present disclosure, in the oligonucleotide-conjugated compound, the 3′-end of the sense strand of the Nu is connected to a phosphate group.

In the double-stranded oligonucleotide represented by Nu, the sense strand comprises a nucleotide sequence of at least 17, at least 18, or at least 19 consecutive nucleotides in any one of the sequences of SEQ ID NO:1, 3, 5, 7, 9, or 11, or comprises a nucleotide sequence having 1, 2, or 3 nucleotide differences from the consecutive nucleotides; and/or the antisense strand comprises a nucleotide sequence of at least 17, at least 18, or at least 19 consecutive nucleotides in any one of the sequences of SEQ ID NO: 2, 4, 6, 8, 10, or 12, or comprises a nucleotide sequence having 1, 2, or 3 nucleotide differences from the consecutive nucleotides.

In some specific embodiments of the present disclosure, in the double-stranded oligonucleotide represented by Nu, in the direction of 5′-3′, the antisense strand comprises the 1st-19th consecutive nucleotides in any one of the sequences of SEQ ID NO: 2, 4, 6, 8, 10, or 12, or comprises a nucleotide sequence having 1, 2, or 3 nucleotide differences from the consecutive nucleotides.

In some specific embodiments of the present disclosure, the sense strand of the double-stranded oligonucleotide represented by Nu comprises any one of the sequences shown in SEQ ID NO. 1, 3, 5, 7, 9 or 11, or a nucleotide sequence having 1 or 2 nucleotide differences from any of the above sequences.

In some specific embodiments of the present disclosure, the antisense strand of the double-stranded oligonucleotide represented by Nu comprises any one of the sequences shown in SEQ ID NO. 2, 4, 6, 8, 10, or 12, or a nucleotide sequence having 1 or 2 nucleotide differences from any of the above sequences.

In some specific embodiments of the present disclosure, the sense strand of the double-stranded oligonucleotide represented by Nu comprises any one of the sequences shown in SEQ ID NO. 1, 3, 5, 7, 9, or 11, and the antisense strand of the double-stranded oligonucleotide represented by Nu comprises any one of the sequences shown in SEQ ID NO. 2, 4, 6, 8, 10, or 12.

In some specific embodiments of the present disclosure, the double-stranded oligonucleotide is selected from one or more of the following groups:
1) the antisense strand has the nucleotide sequence shown in SEQ ID NO. 2 or a nucleotide sequence having 1 or 2 nucleotide differences therefrom; the sense strand has the nucleotide sequence shown in SEQ ID NO. 1 or a nucleotide sequence having 1 or 2 nucleotide differences therefrom;
2) the antisense strand has the nucleotide sequence shown in SEQ ID NO. 4 or a nucleotide sequence having 1 or 2 nucleotide differences therefrom; the sense strand has the nucleotide sequence shown in SEQ ID NO. 3 or a nucleotide sequence having 1 or 2 nucleotide differences therefrom;
3) the antisense strand has the nucleotide sequence shown in SEQ ID NO. 6 or a nucleotide sequence having 1 or 2 nucleotide differences therefrom; the sense strand has the nucleotide sequence shown in SEQ ID NO. 5 or a nucleotide sequence having 1 or 2 nucleotide differences therefrom;
4) the antisense strand has the nucleotide sequence shown in SEQ ID NO. 8 or a nucleotide sequence having 1 or 2 nucleotide differences therefrom; the sense strand has the nucleotide sequence shown in SEQ ID NO. 7 or a nucleotide sequence having 1 or 2 nucleotide differences therefrom;
5) the antisense strand has the nucleotide sequence shown in SEQ ID NO. 10 or a nucleotide sequence having 1 or 2 nucleotide differences therefrom; the sense strand has the nucleotide sequence shown in SEQ ID NO. 9 or a nucleotide sequence having 1 or 2 nucleotide differences therefrom;
6) the antisense strand has the nucleotide sequence shown in SEQ ID NO. 12 or a nucleotide sequence having 1 or 2 nucleotide differences therefrom; the sense strand has the nucleotide sequence shown in SEQ ID NO. 11 or a nucleotide sequence having 1 or 2 nucleotide differences therefrom.

In some specific embodiments of the present disclosure, the double-stranded oligonucleotide is selected from one or more of the following:
1) the antisense strand has the nucleotide sequence shown in SEQ ID NO. 2, and the sense strand has the nucleotide sequence shown in SEQ ID NO. 1;
2) the antisense strand has the nucleotide sequence shown in SEQ ID NO. 4, and the sense strand has the nucleotide sequence shown in SEQ ID NO. 3;
3) the antisense strand has the nucleotide sequence shown in SEQ ID NO. 6, and the sense strand has the nucleotide sequence shown in SEQ ID NO. 5;
4) the antisense strand has the nucleotide sequence shown in SEQ ID NO. 8, and the sense strand has the nucleotide sequence shown in SEQ ID NO. 7;
5) the antisense strand has the nucleotide sequence shown in SEQ ID NO. 10, and the sense strand has the nucleotide sequence shown in SEQ ID NO. 9;
6) the antisense strand has the nucleotide sequence shown in SEQ ID NO. 11, and the sense strand has the nucleotide sequence shown in SEQ ID NO. 12.

In some specific embodiments of the present disclosure, the double-stranded oligonucleotide is selected from:
the antisense strand has the nucleotide sequence shown in SEQ ID NO. 10,
and the sense strand has the nucleotide sequence shown in SEQ ID NO. 9.

In some embodiments of the present disclosure, the siRNA can also comprise modified nucleotides as needed. The modified nucleotides will not cause a significant weakening or loss of the siRNA's function to inhibit the expression of AGT gene. Currently, there are various ways in the art that can be used to modify siRNA, including, for example, backbone modifications (such as phosphate group modifications), ribose group modifications and base modifications. In some embodiments of the present disclosure, at least one nucleotide in the sense strand or the antisense strand of the siRNA is a modified nucleotide. For example, the modified nucleotide is a ribose group and a nucleotide group with an optionally modified phosphate group, but it is not limited to this. In some specific embodiments of the present disclosure, each nucleotide in the double-stranded oligonucleotide is independently selected from modified or unmodified nucleotides.

In some specific embodiments of the present disclosure, substantially all of the nucleotides in the double-stranded oligonucleotide are independently selected from modified nucleotides. Wherein, “substantially all of the nucleotides are selected from modified nucleotides” means that most but not all of the nucleotides in the double-stranded oligonucleotide are modified, and may comprise no more than 5, 4, 3, 2, or 1 unmodified nucleotide(s).

In some specific embodiments of the present disclosure, all nucleotides in the double-stranded oligonucleotide are independently selected from modified nucleotides.

Wherein, the structural formula of the nucleotide is:

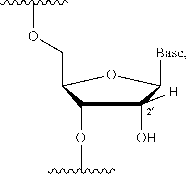

Base represents a nucleoside base, and the nucleoside base on each nucleotide is independently selected from uracil U, thymine T, cytosine C, adenine A, or guanine G.

In some specific embodiments of the present disclosure, each nucleotide in the double-stranded oligonucleotide is independently selected from the following modified nucleotides:
2′-fluoro-modified nucleotides, 2′-deoxy-modified nucleotides, 2′-O-methyl-modified nucleotides, 2′-O—(CH₂)ₓ—O—Rₘ-modified nucleotides, 2′-O—Si(Rₙ)₃-modified nucleotides, 2′-amino-modified nucleotides, abasic nucleotides or nucleotide analogs; the nucleotide analogs are selected from one or more of peptide nucleic acid (PNA), morpholino (MNA), bridged nucleic acid (BNA), locked nucleic acid (LNA), glycol nucleic acid/glycerol nucleic acid (GNA), threose nucleic acid (TNA) or unlocked nucleic acid (UNA).

Wherein, x is selected from 1 or 2, Rₘ is selected from optionally substituted C₁₋₆ alkyl or optionally substituted C₁₋₆ alkoxy, if Rₘ comprises a substituent, the substituent is selected from halogen, C₁₋₆ alkoxy, hydroxyl or amino.

Rₙ is independently selected from optionally substituted C₁₋₆ alkyl.

In the present disclosure, 2′-O—(CH₂)ₓ—Rₘ-modified nucleotides mean nucleotides in which the hydrogen atom on the hydroxyl at the 2′-position of the ribose group is substituted by —(CH₂)ₓ—Rₘ. Wherein, when x is selected from 1, the 2′-O—(CH₂)ₓ—Rₘ-modified nucleotides are selected from 2′-O-ethoxyethyl-modified nucleotides or 2′-O-2,2,2-trifluoroethoxymethyl-modified nucleotides. When x is selected from 2, the 2′-O—(CH₂)ₓ—Rₘ-modified nucleotides are selected from 2′-O-methoxyethyl-modified nucleotides (also referred to 2′-O-moe-modified nucleotides).

In some specific embodiments of the present disclosure, the 2′-O—(CH₂)ₓ—O—Rₘ-modified nucleotides are selected from 2′-O-methoxyethyl-modified nucleotides or 2′-O-ethoxyethyl-modified nucleotides.

In the present disclosure, “2′-O—Si(Rₙ)₃-modified nucleotides” means nucleotides formed by the substitution of the hydrogen atom on the hydroxyl at the 2′-position of the ribose group of the nucleotide with —Si(Rₙ)₃. Exemplarily, 2′-O-TBDMS-modified nucleotides, 2′-O-TIPS-modified nucleotides or 2′-O-TOM-modified nucleotides; wherein, the structural formula of TBDMS is

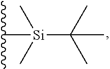 [SMILES: *[Si](C)(C)C(C)(C)C]

the structural formula of TIPS is

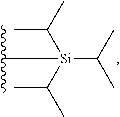 [SMILES: *[Si](C(C)C)(C(C)C)C(C)C]

and the structural formula of TOM is

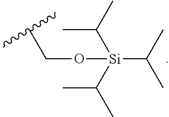 [SMILES: *CO[Si](C(C)C)(C(C)C)C(C)C]

In some specific embodiments of the present disclosure, the 2′-O—Si(Rₙ)₃-modified nucleotides are selected from 2′-O-TBDMS-modified nucleotides, 2′-O-TIPS-modified nucleotides or 2′-O-TOM-modified nucleotides.

In some specific embodiments of the present disclosure, the double-stranded oligonucleotide comprises at least one 2′-O-methoxyethyl-modified nucleotide.

According to some specific embodiments of the present disclosure, the sense strand is 17-21 nucleotides in length, and the antisense strand is 19-23 nucleotides in length.

According to some specific embodiments of the present disclosure, the sense strand is 19 nucleotides in length, and the antisense strand is 21 nucleotides in length.

In some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from nucleotides other than 2′-fluoro-modified nucleotides; at least four nucleotides among the nucleotides at positions 2nd, 6th, 9th-12th, 14th and 16th of the nucleotide sequence in the antisense strand are selected from 2′-fluoro-modified nucleotides, and the nucleotides at the remaining positions are independently selected from nucleotides other than 2′-fluoro-modified nucleotides.

According to some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from 2′-O-methyl-modified nucleotides or 2′-O-methoxyethyl-modified nucleotides.

According to some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from 2′-O-methyl-modified nucleotides.

According to some specific embodiments of the present disclosure, in the direction from the 5′-end to the 3′-end, at least three nucleotides among the nucleotides at positions 7th-10th of the nucleotide sequence in the sense strand are selected from 2′-fluoro-modified nucleotides, the nucleotides at positions 12th and/or 18th are selected from 2′-O-methoxyethyl-modified nucleotides, and the nucleotides at the remaining positions are independently selected from 2′-O-methyl-modified nucleotides.

According to some specific embodiments of the present disclosure, in the direction from the 5′-end to the 3′-end, the nucleotides at positions 7th-10th in the nucleotide sequence of the sense strand are selected from 2′-fluoro-modified nucleotides. The nucleotides at positions 12th and/or 18th are selected from 2′-O-methoxyethyl-modified nucleotides, and the nucleotides at the remaining positions are independently selected from 2′-O-methyl-modified nucleotides.

According to some specific embodiments of the present disclosure, at least four nucleotides among the nucleotides at positions 2nd, 6th, 9th-12th, 14th and 16th in the nucleotide sequence of the antisense strand are selected from 2′-fluoro-modified nucleotides, and the nucleotides at the remaining positions are selected from 2′-O-methyl-modified nucleotides or 2′-O-methoxyethyl-modified nucleotides.

According to some specific embodiments of the present disclosure, at least four nucleotides among the nucleotides at positions 2nd, 6th, 9th-12th, 14th and 16th in the nucleotide sequence of the antisense strand are selected from 2′-fluoro-modified nucleotides, and the nucleotides at positions 2nd, 6th, 14th and 16th are selected from 2′-fluoro-modified nucleotides. The nucleotides at the remaining positions are selected from 2′-O-methyl-modified nucleotides or 2′-O-methoxyethyl-modified nucleotides.

According to some specific embodiments of the present disclosure, at least four nucleotides among the nucleotides at positions 2nd, 6th, 9th-12th, 14th and 16th in the nucleotide sequence of the antisense strand are selected from 2′-fluoro-modified nucleotides, and the nucleotides at positions 2nd, 6th, 14th and 16th are selected from 2′-fluoro-modified nucleotides. The nucleotides at positions 8th and/or 15th are selected from 2′-O-methoxyethyl-modified nucleotides, and the nucleotides at the remaining positions are selected from 2′-O-methyl-modified nucleotides.

According to some specific embodiments of the present disclosure, in the direction from the 5′-end to the 3′-end, at least three nucleotides among the nucleotides at positions 7th-10th in the nucleotide sequence of the sense strand are selected from 2′-fluoro-modified nucleotides, and the nucleotides at the remaining positions are independently selected from 2′-O-methyl-modified nucleotides or 2′-O-methoxyethyl-modified nucleotides. At least four nucleotides among the nucleotides at positions 2nd, 6th, 9th-12th, 14th and 16th in the nucleotide sequence of the antisense strand are selected from 2′-fluoro-modified nucleotides, and the nucleotides at the remaining positions are selected from 2′-O-methyl-modified nucleotides or 2′-O-methoxyethyl-modified nucleotides.

According to some specific embodiments of the present disclosure, each nucleotide of the double-stranded oligonucleotide is independently selected from modified nucleotides, wherein the modification of the sense strand and the antisense strand is selected from any one of the following (1)-(15):
(1) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 9th, 14th and 16th in the nucleotide sequence of the antisense strand are selected from 2′-fluoro-modified nucleotides, and the nucleotides at the remaining positions are selected from 2′-O-methyl-modified nucleotides;
(2) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, and the nucleotides at the remaining positions are selected from 2′-O-methyl-modified nucleotides;
(3) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 11th, 14th and 16th in the nucleotide sequence of the antisense strand are selected from 2′-fluoro-modified nucleotides, and the nucleotides at the remaining positions are selected from 2′-O-methyl-modified nucleotides;
(4) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 12th, 14th and 16th in the nucleotide sequence of the antisense strand are selected from 2′-fluoro-modified nucleotides, and the nucleotides at the remaining positions are selected from 2′-O-methyl-modified nucleotides;
(5) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 9th, 14th and 16th in the nucleotide sequence of the antisense strand are selected from 2′-fluoro-modified nucleotides, the nucleotide at position 15th is selected from 2′-O-methoxyethyl modified nucleotides, and the nucleotides at the remaining positions are selected from 2′-O-methyl-modified nucleotides;
(6) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, the nucleotide at position 15th is selected from 2′-O-methoxyethyl modified nucleotides, and the nucleotides at the remaining positions are selected from 2′-O-methyl-modified nucleotides;
(7) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 11th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(8) in the direction from the 5′-end to the 3′-end, the nucleotides at positions 7th-10th in the nucleotide sequence of the sense strand are selected from 2′-fluoro-modified nucleotides, and the nucleotides at the remaining positions are selected from 2′-O-methyl-modified nucleotides; the nucleotides at positions 2nd, 6th, 12th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(9) in the direction from the 5′-end to the 3′-end, the nucleotides at positions 7th-10th in the nucleotide sequence of the sense strand are selected from 2′-fluoro-modified nucleotides, the nucleotide at position 12th is selected from 2′-O-methoxyethyl-modified nucleotides, and the nucleotides at the remaining positions are selected from 2′-O-methyl-modified nucleotides; the nucleotides at positions 2nd, 6th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(10) in the direction from the 5′-end to the 3′-end, the nucleotides at positions 7th-10th in the nucleotide sequence of the sense strand are selected from 2′-fluoro-modified nucleotides, the nucleotide at position 12th is selected from 2′-O-methoxyethyl-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(11) in the direction from the 5′-end to the 3′-end, the nucleotides at positions 7th-10th in the nucleotide sequence of the sense strand are selected from 2′-fluoro-modified nucleotides, the nucleotide at position 12th is selected from 2′-O-methoxyethyl-modified nucleotides, and the nucleotides at the remaining positions are selected from 2′-O-methyl-modified nucleotides; the nucleotides at positions 2nd, 6th, 11th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(12) In the direction from the 5′-end to the 3′-end, the nucleotides at positions 7th-10th in the nucleotide sequence of the sense strand are selected from 2′-fluoro-modified nucleotides, the nucleotide at position 18 is selected from 2′-O-methoxyethyl-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(13) in the direction from the 5′-end to the 3′-end, the nucleotides at positions 7th-10th in the nucleotide sequence of the sense strand are selected from 2′-fluoro-modified nucleotides, the nucleotide at position 18 is selected from 2′-O-methoxyethyl-modified nucleotides, and the nucleotides at the remaining positions are selected from 2′-O-methyl-modified nucleotides; the nucleotides at positions 2nd, 6th, 11th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides;
(14) in the direction from the 5′-end to the 3′-end, the nucleotides at positions 7th-10th in the nucleotide sequence of the sense strand are selected from 2′-fluoro-modified nucleotides, the nucleotide at position 18 is selected from 2′-O-methoxyethyl-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, the nucleotide at position 8 is selected from 2′-O-methoxyethyl-modified nucleotides, and the nucleotides at the remaining positions are selected from 2′-O-methyl-modified nucleotides; or
(15) in the direction from the 5′-end to the 3′-end, the nucleotides at positions 7th-10th in the nucleotide sequence of the sense strand are selected from 2′-fluoro-modified nucleotides, the nucleotides at positions 12th and 18th are selected from 2′-O-methoxyethyl-modified nucleotides, and the nucleotides at the remaining positions are selected from 2′-O-methyl-modified nucleotides; the nucleotides at positions 2nd, 6th, 10th, 14th and 16th in the nucleotide sequence of the antisense strand are selected from 2′-fluoro-modified nucleotides, the nucleotide at position 15th is selected from 2′-O-methoxyethyl-modified nucleotides, and the nucleotides at the remaining positions are selected from 2′-O-methyl-modified nucleotides.

In some specific embodiments of the present disclosure, the sense strand and/or the antisense strand independently comprises one or more phosphorothioate internucleotide bonds.

In some specific embodiments of the present disclosure, in the direction from the 5′-end to the 3′-end, at least one of the linkages between the nucleotides of the sense strand is a phosphorothioate linkage:
(1) the linkage between the first and the second nucleotides at the 5′-end of the sense strand;
(2) the linkage between the second and the third nucleotides at the 5′-end of the sense strand.
In some specific embodiments of the present disclosure, in the direction from the 5′-end to the 3′-end, at least one of the linkages between the following nucleotides of the antisense strand is a phosphorothioate linkage:
(1) the linkage between the first and the second nucleotides at the 5′-end of the antisense strand;
(2) the linkage between the second and the third nucleotides at the 5′-end of the antisense strand;
(3) the linkage between the first and the second nucleotides at the 3′-end of the antisense strand;
(4) the linkage between the second and the third nucleotides at the 3′-end of the antisense strand.

In some optional embodiments of the present disclosure, the sense strand or the antisense strand comprises a 3′-overhang with at least one nucleotide.

In some optional embodiments of the present disclosure, the antisense strand comprises a 3′-overhang with at least one nucleotide.

In some optional embodiments of the present disclosure, the antisense strand comprises a 3′-overhang with two nucleotides.

In some specific embodiments of the present disclosure, each nucleotide in the double-stranded oligonucleotide is selected from modified nucleotides.

In some specific embodiments of the present disclosure, the double-stranded oligonucleotide is selected from at least one group of the following groups.

| | Sense strand (5′-3′) | Antisense strand (5′-3′) |
|---|---|---|
| group 1 | CmsAmsAmGmUmUmGfAfGf | AmsAfsUmUmUmUfUmGmUmU |
| | AfAmCmAmAmAmAmAmUm | fCmUmCmAfA(moe)CfUmUmGm |
| | Um | sAmsAm |
| group 2 | GmsUmsUmUmUmAmAfAfAf | UmsAfsUmAmCmUfUmUmAmA |
| | UfUmAmAmAmGmUmAmUm | mUfUmUmUfA(moe)AfAmAmC |
| | Am | msCmsCm |
| group 3 | AmsAmsGmUmAmUmAfCfAf | AmsAfsUmGmCmAfAmAmAmAf |
| | UfUmUmUmUmGmCmAmUm | UmGmUmAfUmAfCmUmUmsU |
| | Um | msAm |
| group 4 | AmsAmsGmUmAmUmAfCfAf | AmsAfsUmGmCmAfAmAmAmAf |
| | UfUmUmUmUmGmCmAmUm | UmGmUmAfT(moe)AfCmUmUm |
| | Um | sUmsAm |

In some specific embodiments of the present disclosure, the double-stranded oligonucleotide is selected from the following group:

| | Sense strand (5′-3′) | Antisense strand (5′-3′) |
|---|---|---|
| group 2 | GmsUmsUmUmUmAmAfAfAf | UmsAfsUmAmCmUfUmUmAmA |
| | UfUmAmAmAmGmUmAmUm | mUfUmUmUfA(moe)AfAmAmC |
| | Am | msCmsCm |

wherein, C represents cytidine-3′-phosphate, G represents guanosine-3′-phosphate, U represents uridine-3′-phosphate, A represents adenosine-3′-phosphate, and T represents thymidine-3′-phosphate; m indicates that the nucleotide adjacent to the left of the letter m is a 2′-O-methyl-modified nucleotide; f indicates that the nucleotide adjacent to the left of the letter f is a 2′-fluoro-modified nucleotide; (moe) indicates that the nucleotide adjacent to the left of the combination identifier (moe) is a 2′-O-methoxyethyl-modified nucleotide; s indicates that the two adjacent nucleotides are connected by a phosphorothioate diester bond.

In some specific embodiments of the present disclosure, the oligonucleotide-conjugated compound is selected from one or more groups in Table 6.

In some specific embodiments of the present disclosure, the oligonucleotide-conjugated compound comprises one or more of the following groups:

| | Sense strand (5′-3′) | Antisense strand (5′-3′) |
|---|---|---|
| RZ003062 | CmsAmsAmGmUmUmGfAfG | AmsAfsUmUmUmUfUmGmUmU |
| | fAfAmCmAmAmAmAmAmU | fCmUmCmAfA(moe)CfUmUmGm |
| | mUm_(CR01008 × 3) | sAmsAm |
| RZ003064 | GmsUmsUmUmUmAmAfAf | UmsAfsUmAmCmUfUmUmAmA |
| | AfUfUmAmAmAmGmUmA | mUfUmUmUfA(moe)AfAmAmC |
| | mUmAm_(CR01008 × 3) | msCmsCm |
| RZ003065 | AmsAmsGmUmAmUmAfCfA | AmsAfsUmGmCmAfAmAmAmAf |
| | fUfUmUmUmUmGmCmAmU | UmGmUmAfUmAfCmUmUmsU |
| | mUm_(CR01008 × 3) | msAm |
| RZ003066 | AmsAmsGmUmAmUmAfCfA | AmsAfsUmGmCmAfAmAmAmAf |
| | fUfUmUmUmUmGmCmAmU | UmGmUmAfT(moe)AfCmUmUm |
| | mUm_(CR01008 × 3) | sUmsAm |
| RZ003089 | UmsCmsAmAmCmUmGfGfA | AmsGfsUmUmUmCfUmUmCfAm |
| | fUfGmAmAmGmAmAmAmC | UmCmCmAfG(moe)UfUmGmAm |
| | mUm_(CR01008 × 3) | sGmsGm |

For an exemplary explanation, “_(CR01008×3)” means that the ligand represented by the Formula (CR01008×3) is conjugated to the 3′-end of the sense strand.

In some specific embodiments of the present disclosure, the oligonucleotide-conjugated compound comprises the following group

| | Sense strand (5′-3′) | Antisense strand (5′-3′) |
|---|---|---|
| RZ003064 | GmsUmsUmUmUmAmAfAf | UmsAfsUmAmCmUfUmUmAmA |
| | AfUfUmAmAmAmGmUmA | mUfUmUmUfA(moe)AfAmAmC |
| | mUmAm_(CR01008 × 3) | msCmsCm |

### Pharmaceutical Compositions

In the second aspect of the present disclosure, the present disclosure provides a pharmaceutical composition. The pharmaceutical composition comprises the oligonucleotide-conjugated compound according to the first aspect of the present disclosure.

In some optional embodiments of the present disclosure, the pharmaceutical composition further comprises one or more pharmaceutically acceptable excipients.

The pharmaceutical composition of the present disclosure includes formulations suitable for parenteral administration.

The formulations can conveniently be presented in unit-dosage forms and can be prepared by any method well-known in the pharmaceutical field. The amount of the active ingredient, which can be combined with excipient materials to prepare a single-dose form, is generally the amount of siRNA that produces a therapeutic effect. Generally, in percentage terms, the amount ranges from about 1% to about 99% of the active ingredient, preferably from about 5% to about 70%, and most preferably from about 10% to about 30%.

### Uses

In the third aspect of the present disclosure, the present disclosure provides a use of the oligonucleotide-conjugated compound according to the first aspect of the present disclosure or the pharmaceutical composition according to the second aspect of the present disclosure in the manufacture of a medicament for alleviating, preventing and/or treating a disease or disorder mediated by AGT gene.

In some optional embodiments of the present disclosure, the disease or disorder mediated by the AGT gene includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

### Kits

In the fourth aspect of the present disclosure, the present disclosure provides a kit comprising the oligonucleotide-conjugated compound according to the first aspect of the present disclosure or the pharmaceutical composition according to the second aspect of the present disclosure.

### Methods for Inhibiting the Expression of AGT Gene

In the fifth aspect of the present disclosure, the present disclosure provides a method for inhibiting the expression of AGT gene, which comprises administering to a subject the oligonucleotide-conjugated compound according to the first aspect of the present disclosure or the pharmaceutical composition according to the second aspect of the present disclosure.

In some specific embodiments of the present disclosure, a method for inhibiting the expression of AGT gene in a cell in vitro comprises contacting the cells with the oligonucleotide-conjugated compound according to the first aspect of the present disclosure or the pharmaceutical composition according to the second aspect of the present disclosure.

In some optional embodiments of the present disclosure, the inhibition of the expression of AGT gene in a cell is to maintain the cells for a duration sufficient to achieve the degradation of the mRNA transcripts of the AGT gene.

### Treatment Methods for Diseases

In the sixth aspect of the present disclosure, the present disclosure provides a method for alleviating, preventing and/or treating a disease or disorder mediated by AGT, which comprises administering to a subject the oligonucleotide-conjugated compound according to the first aspect of the present disclosure or the pharmaceutical composition according to the second aspect of the present disclosure.

In some specific embodiments of the present disclosure, the subject is human.

In some specific embodiments of the present disclosure, the disease or disorder mediated by AGT includes the diseases associated with the mRNA level of the expression of AGT gene.

In some specific embodiments of the present disclosure, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

The effective amount of the oligonucleotide-conjugated compound or the pharmaceutical composition according to the present disclosure can vary depending on the mode of administration, the severity of the disease to be treated, etc. The selection of the preferred effective amount can be determined by those ordinary skilled in the art based on various factors (e.g., through clinical trials). The factors include, but are not limited to: the pharmacokinetic parameters of the active ingredient, such as bioavailability, metabolism, half-life, etc.; the severity of the disease for which the patient needs to be treated, the patient's body weight, the patient's immune status, the route of administration, etc. For example, depending on the urgency of the treatment situation, several divided doses can be administered daily, such as four times a day, three times a day, twice a day, once a day, or once every other day, or the amount of several doses administered daily can be proportionally reduced.

Administration to a subject can be carried out via any suitable route well-known in the art. The routes include, but are not limited to: oral or parenteral routes, including intravenous administration, intramuscular administration, subcutaneous administration, transdermal administration, airway administration (aerosol), pulmonary administration, nasal administration, rectal administration, and topical administration (including buccal and sublingual administration). Intravenous injection administration is preferred.

The oligonucleotide-conjugated compound and the pharmaceutical compositions provided by the present disclosure can induce silencing complex (RISC)-mediated cleavage of RNA transcripts of the AGT gene, inhibit the expression of AGT gene, and thus help to treat or prevent a disease or disorder mediated by angiotensinogen (AGT) gene.

To make the objectives, technical solutions, and advantages of the present disclosure clearer, the implementation schemes of the present disclosure will be described in further detail below in conjunction with examples.

Unless otherwise specified, reagents used in the preparation of compounds in the present disclosure were all purchased from Beijing Ouhe Technology Co., Ltd., and the information of the main reagents is shown in Table 1.

TABLE 1
Main Reagents

| | | CAS |
|---|---|---|
| Reagent Name | Abbreviation | Number |
| Lithium aluminum hydride | LiAlH₄ | 16853- |
| | | 85-3 |
| Wet palladium on carbon (10 wt% loading) | Pd/C | — |
| Palladium hydroxide on carbon (10 wt% loading) | Pd(OH)₂/C | — |
| Benzotriazol-N,N,N′,N′-tetramethyluronium | HBTU | 94790- |
| hexafluorophosphate | | 37-1 |
| 2-(7-Azabenzotriazol-1-yl)-N,N,N′,N′- | HATU | 14889 |
| tetramethyluronium hexafluorophosphate | | 3-10-1 |
| 4,4′-Dimethoxytrityl chloride/4,4′- | DMTrCl | 40615- |
| Dimethoxytriphenylmethyl chloride | | 36-9 |
| Bis(diisopropylamino)(2-cyanoethoxy)phosphine | — | 10269 |
| | | 1-36-1 |
| 4,5-Dicyanoimidazole | DCI | 1122- |
| | | 28-7 |
| 4-Dimethylaminopyridine | DMAP | 1122- |
| | | 58-3 |
| Aminoalkyl-CPG(Model C3006-1000) | 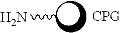 | — |
| 1,4-Dioxane solution of 4M hydrochloric acid | — | — |
| Trans-4-(Boc-amino)cyclohexanecarbaldehyde | — | 18130 |
| | | 8-57-6 |
| N-Benzyloxycarbonyl-4-aminobutyric acid | — | 5105- |
| | | 78-2 |
| 5-[[(2R,3R,4R,5R,6R)-3-Acetamido-4,5-diacetoxy- | Compound 4 | 11594 |
| 6-(acetoxymethyl)-2- | | 08-54- |
| tetrahydropyranylloxy lpentanoic acid | | 4 |

wherein, CPG represents a Controlled Pore Glass carrier.

Unless otherwise specified, the reagents, consumables, and equipment used in the biological detection experiments of the present disclosure are all commercially available products. Wherein, the main reagents and consumables and their sources are shown in Table 2, and the main equipment and their sources are shown in Table 3.

TABLE 2
Main reagents and consumables

| Name | Manufacturer |
|---|---|
| 1 × PBS | Zhongke Maichen (Beijing) |
| | Technology Co., Ltd. |
| DMEM Medium | Zhongke Maichen (Beijing) |
| | Technology Co., Ltd. |
| Opti-MEM ™ Medium | Gibco |
| Serum | Sigema |
| Trypsin | Zhongke Maichen (Beijing |
| | Technology Co., Ltd. |
| Double Antibiotics | BBI |
| Lipofectamine RNAiMax | Invitrogen |
| Nucleic Acid Extraction or | Zhejiang Hanwei Technology |
| Purification Kit | Co., Ltd. |
| RevertAid First Strand | Thermo Fisher Scientific |
| cDNA Synthesis Kit | |
| TaqMan Fast Advanced Master Mix | Thermo Fisher Scientific |
| SYBR Select Master Mix | Thermo Fisher Scientific |
| Hanwei RNA Extraction Kit | Zhejiang Hanwei Technology Co., |
| | Ltd. |
| RNALater | Thermo Fisher Scientific |
| Human Angiotensinogen Kit | IBL |
| Rat Ang-II ELISA Kit | ZCIBIO Technology Co., Ltd |
| Zoletil 50 | Virbac (France) |

TABLE 3
Main equipment

| Name | Manufacturer |
|---|---|
| Automatic nucleic acid | Zhejiang Hanwei Technology |
| extractor | Co., Ltd. |
| High-speed refrigerated | Eppendorf |
| centrifuge | |
| Carbon dioxide incubator | Thermo Fisher Scientific |
| Biological safety cabinet | Heal Force Bio-meditech |
| | Holdings Limited |
| Constant temperature water | Shanghai Boxun Medical |
| bath | Biological Instrument Corp. |
| Automatic cell counter | Alit Biotech (Shanghai) Co., |
| | Ltd. |
| Inverted microscope | Olympus |
| NANODROP OneC | Thermo Fisher Scientific |
| Gradient PCR amplifier | Eppendorf |
| CFX Opus 384 | Bio-Rad |
| LightCycler 480 | Roche |
| Real-time fluorescence | Thermo Fisher Scientific |
| quantitative (RT-PCR) instrument | |
| QuantStudio TM3 | |
| Gel imaging system | Shanghai Tanon Life Science |
| | Co., Ltd. |
| Electrophoresis apparatus | BEIJING LIUYI |
| | BIOTECHNOLOGY |
| | CO., LTD. |
| Tissuelyser II automatic | Shanghai Jingxin Industrial |
| tissue homogenizer | Development Co., Ltd |
| Paraffin microtome | Jinhua YIDI Medical |
| | Appliance CO., LTD |
| Tissue dehydrator | Leica |
| Automatic biological tissue | Jinhua YIDI Medical |
| embedding machine | Appliance CO., LTD |
| Automatic modular blood and | SYSMEX |
| body fluid analyzer | |
| Automatic biochemical analyzer | SYSMEX |
| Automatic coagulation analyzer | Mindray |

In the present disclosure, unless otherwise specified, the ratios of the reagents used in the present disclosure are calculated by volume ratio (v/v).

### Preparation of Compounds

### Preparation Example 1: Preparation of Compound CR01008 and Compound CR01008Z

### (1.1) Synthesis of Compound CR01008

In the Preparation Example, the synthetic route for compound CR01008 is as follows:

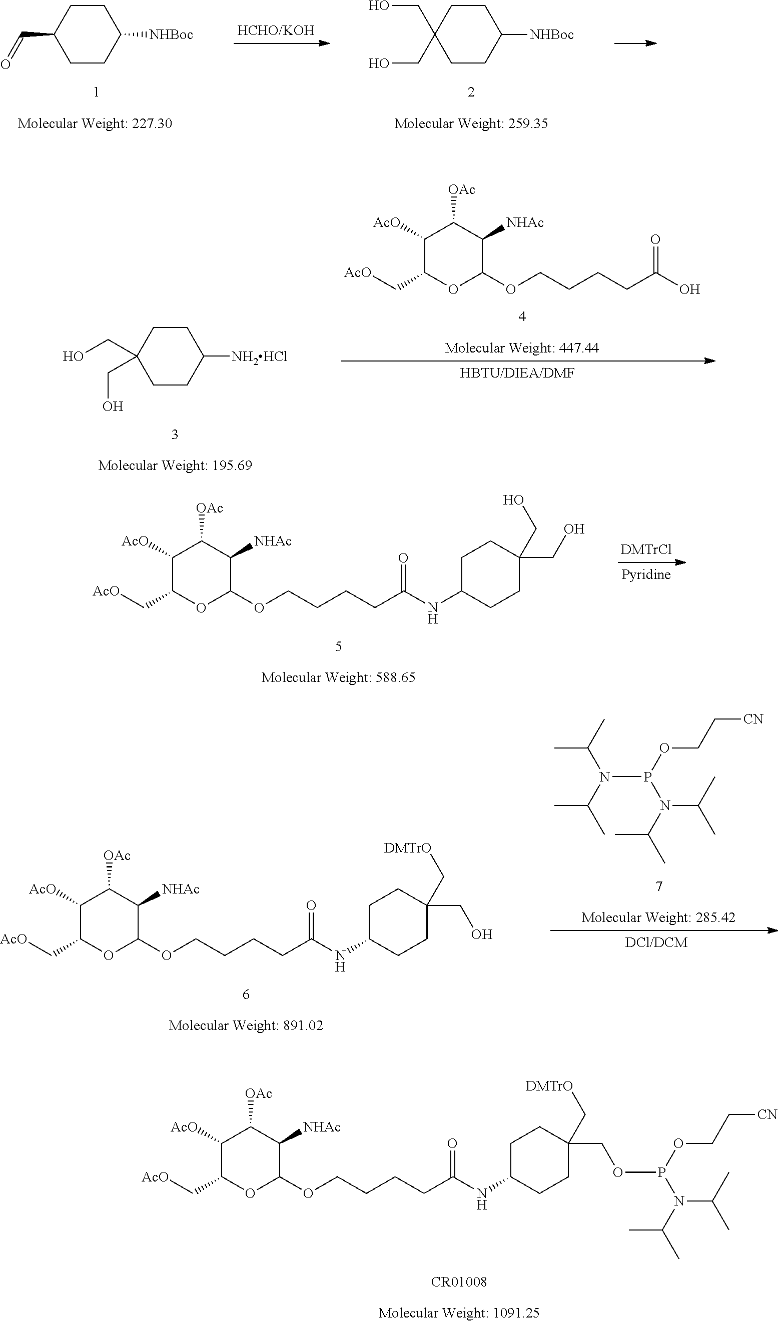

### (1.1.1) Synthesis of Compound 2

Compound 1 (trans-4-(Boc-amino)cyclohexanecarbaldehyde, 10.0 g, 1.0 eq) and aqueous formaldehyde solution (8.9 g, 37% by mass, 2.4 eq) were dissolved in 33 mL of methanol. Then, 13 mL of aqueous KOH solution with a concentration of 45.3% by mass was added dropwise. After the addition was completed, the mixture was stirred at 25° C. for 30 minutes and then warmed to 60° C. and refluxed to react at 60° C. for 2 hours. After the reaction was finished, the reaction liquid was cooled to room temperature and then evaporated under reduced pressure to obtain a white solid crude product. A small amount of water was added to the crude product for slurry, and then the mixture was filtered to obtain compound 2 as a white solid (9 g, yield 78.9%). MS-ESI (m/z)=260 [M+H]⁺.

### (1.1.2) Synthesis of Compound 3

Compound 2 (9 g, 1 eq) obtained according to the preparation method in step (1.1.1) was dissolved in 70 mL of 1,4-dioxane. Then, a 1,4-dioxane solution of hydrogen chloride (45 mL, 4 M) was added. The mixture was stirred at 25° C. for 1 hour. After the reaction was completed, the reaction liquid was evaporated to dryness under reduced pressure to obtain compound 3 as a white solid (6.8 g, yield 100%).

### (1.1.3) Synthesis of Compound 5

Compound 3 (1.8 g, 2.0 eq) obtained according to the preparation method in step (1.1.2), compound 4 (5-[[(2R,3R,4R,5R,6R)-3-acetamido-4,5-diacetoxy-6-(acetoxymethyl)-2-tetrahydropyranyl]oxy]pentanoic acid, 2.1 g, 1.0 eq) and DIEA (N,N-diisopropylethylamine, 3.5 g, 6.0 eq) were dissolved in 15 mL of DMF, then, HBTU (1.9 g, 1.1 eq) was added. The mixture was stirred at 25° C. for 3 hours under a N₂ atmosphere. After the reaction was completed, the reaction liquid was evaporated to dryness under reduced pressure, and purified by reverse-phase chromatography (22% acetonitrile aqueous solution by volume) to obtain compound 5 as a white solid (1.78 g, yield 64.4%). MS-ESI (m/z)=589[M+H]⁺.

### (1.1.4) Synthesis of Compound 6

Compound 5 (1.54 g, 1.0 eq) obtained according to the preparation method in step (1.1.3) was dissolved in 15 mL of pyridine. The reaction system was cooled to 0° C. using an ice-water bath, and DMTrC1 (4,4′-dimethoxytrityl chloride, 1.32 g, 1.5 eq) was added at 0° C. The mixture was reacted at 25° C. for 3 hours, then, 15 mL of methanol was added to the reaction liquid to quench the reaction. After the reaction was completed, the reaction liquid was evaporated to dryness under reduced pressure and purified by reverse-phase chromatography (60% acetonitrile aqueous solution by volume) to obtain compound 6 as a yellow solid (1 g, yield 42.7%). MS-ESI (m/z)=891 [M+H]⁺.

### (1.1.5) Synthesis of Compound CR01008

Compound 6 (1.08 g, 1.0 eq) obtained according to the preparation method in step (1.1.4) was dissolved in 20 mL of anhydrous dichloromethane. DCI (115 mg, 0.8 eq) and compound 7 (bis(diisopropylamino)(2-cyanoethoxy)phosphine, 732 mg, 2.1 eq) were added respectively. The reaction system was purged with nitrogen three times, and then the mixture was stirred at 25° C. for 2 hours. After the reaction was completed, 20 mL of saturated aqueous sodium bicarbonate solution was added to the reaction liquid. The mixture was extracted with 20 mL of dichloromethane three times (3×20 mL). The organic phases were combined and evaporated to dryness under reduced pressure. After being purified by reverse-phase chromatography (72% acetonitrile aqueous solution by volume), the product was dried under vacuum for 12 hours to obtain compound CR01008 as a white powder (1 g, yield 76.0%). MS-ESI (m/z)=1091 [M+Na]⁺.

1H NMR (400 MHz, DMSO-d6) δ 1.05 (d, J=6.7 Hz, 6H). 1.14 (d, J=6.7 Hz, 6H), 1.37-1.17 (m, 5H), 1.60-1.40 (m, 6H), 1.68-1.62 (m, 1H), 1.80 (s, 3H), 1.80 (s, 3H), 1.92 (s, 3H), 2.02 (s, 5H), 2.13 (s, 3H), 2.71 (t, J=5.9 Hz, 2H), 2.79 (d, J=8.4 Hz, 1H), 2.87 (d, J=8.4 Hz, 1H), 3.36 (s, 1H), 3.58-3.39 (m, 3H), 3.69-3.60 (m, 2H), 3.75 (s, 7H), 3.90 (dt, J=11.2, 8.8 Hz, 1H), 4.05 (s, 3H), 4.51 (d, J=8.4 Hz, 1H), 4.99 (dd, J=11.3, 3.4 Hz, 1H), 5.24 (d, J=3.4 Hz, 1H), 5.78 (s, 1H), 6.93-6.87 (m, 4H), 7.35-7.21 (m, 7H), 7.44-7.37 (m, 2H), 7.66 (d, J=7.8 Hz, 1H), 7.84 (d, J=9.2 Hz, 1H).

### (1.2) Synthesis of Compound CR01008Z

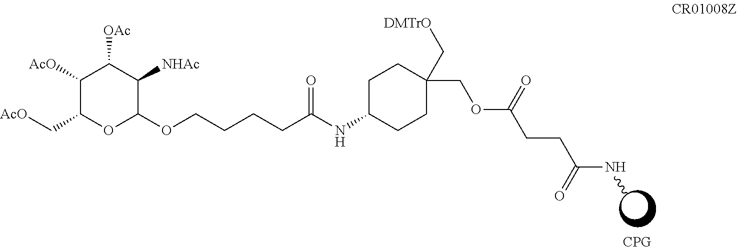

In the Preparation Example, the synthetic route for compound CR01008Z is as follows:

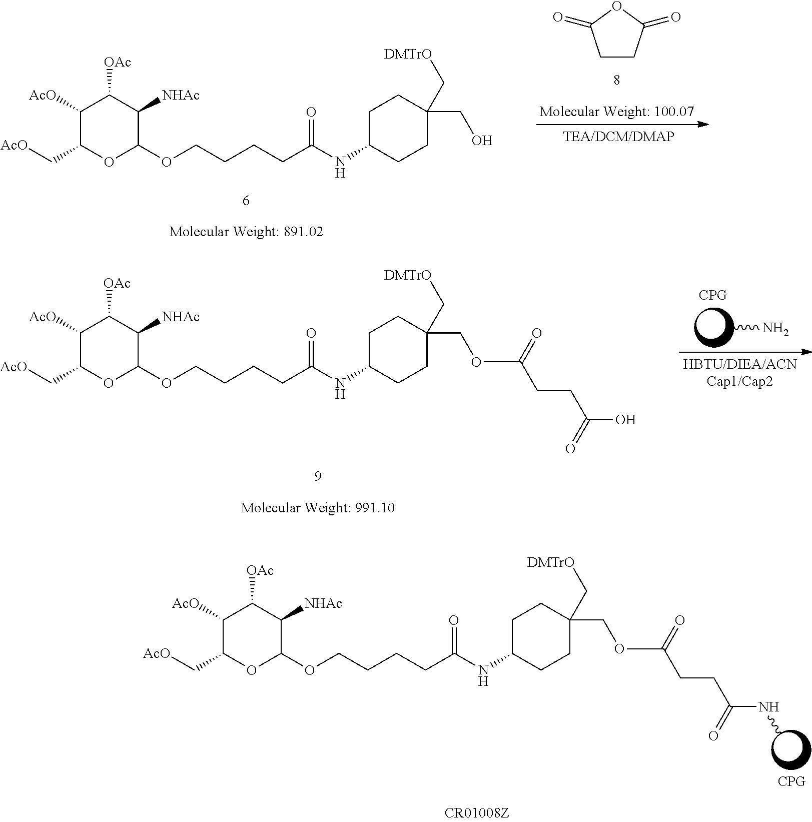

### (1.2.1) Synthesis of Compound 9

Compound 6 (500 mg) obtained according to the preparation method in step (1.1.4) was dissolved in 10 mL of dichloromethane. Compound 8 (succinic anhydride, 112 mg), DMAP (6.8 mg) and TEA (226.2 mg) were added. The reaction system was purged with nitrogen three times and then the mixture was stirred at 25° C. for 16 hours. After flash purification, compound 9 (300 mg, yield 53.6%) was obtained. MS-ESI (m/z)=1013 [M+Na]⁺.

### (1.2.2) Synthesis of Compound CR01008Z

To a 20 mL sample vial, compound 9 (50 mg) obtained according to the preparation method in step (1.2.1), aminoalkyl—CPG (1.25 g, 80 μmol/g, 0.1 mmol), HBTU (27 mg) and DIEA (12 mg) were added. The mixture was reacted on a shaker for 16 hours. After the reaction was completed, the reaction liquid was filtered to obtain a filter cake. The filter cake was washed once with 10 mL of acetonitrile (1×10 mL) and then dried under vacuum. To a 20 mL sample vial, the dried filter cake, DMAP (3 mg), Cap1 (10 mL, 200V) and Cap2 (1 mL, 20V) were added. The mixture was reacted on a shaker for 6 hours. After the reaction was finished, the reaction liquid was filtered to obtain a filter cake. The filter cake was washed once with 10 mL of acetonitrile (1×10 mL) and then dried under vacuum to obtain compound CR01008Z (1.03 g, loading capacity: 20-30 mol/g).

Wherein, Cap1 and Cap2 are capping reagents. Cap1 is a pyridine/acetonitrile mixed solution comprising 20% N-methylimidazole by volume, with a volume ratio of pyridine to acetonitrile of 3:5. Cap2 is a 20% acetic anhydride solution in acetonitrile by volume.

### Preparation Example 2: Preparation of Compound CR01013 and Compound CR01013Z

### (2.1) Preparation of Compound CR01013

In the Preparation Example, the synthetic route for compound CR01013 is as follows:

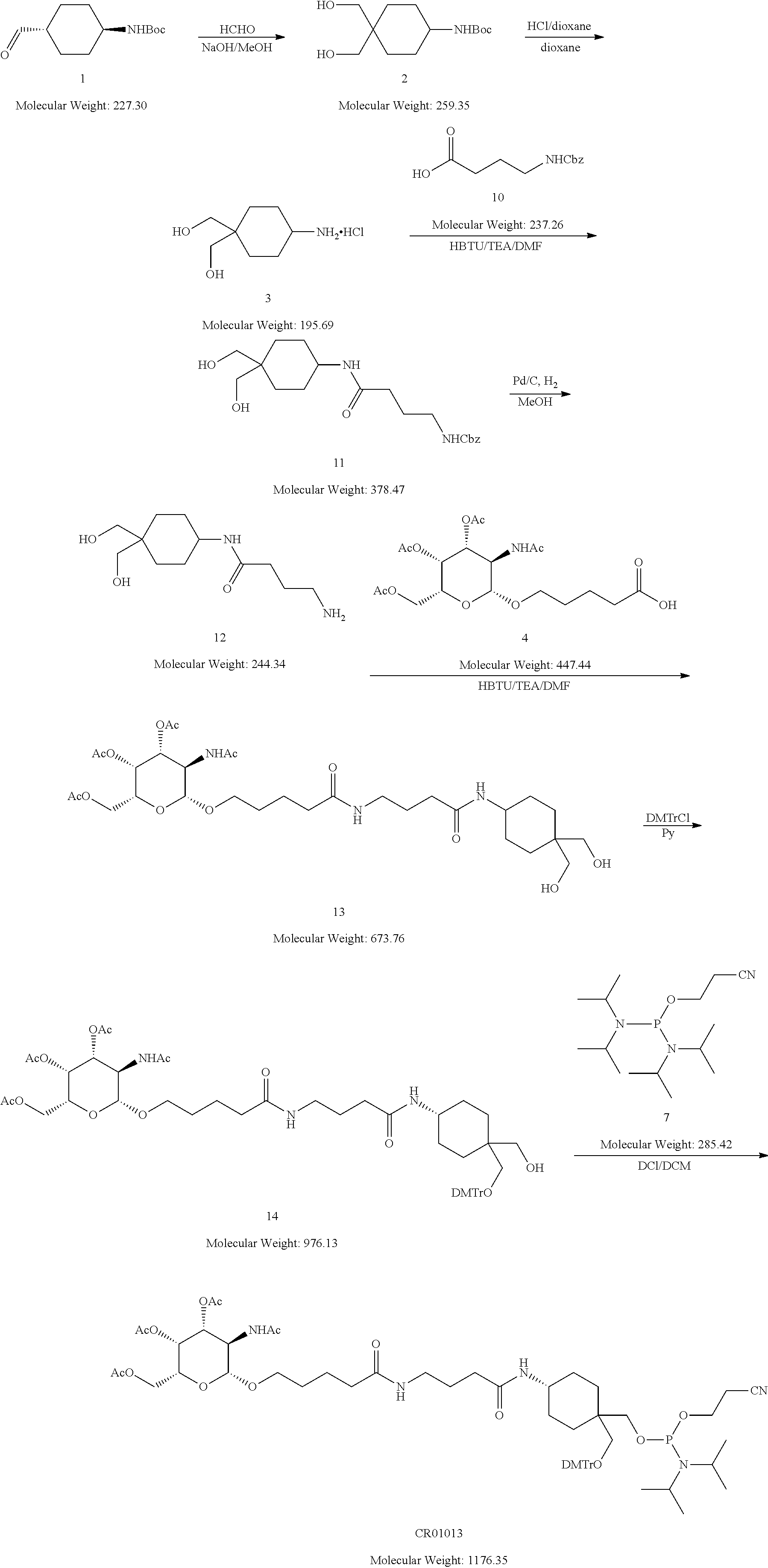

### (2.1.1) Synthesis of Compound 2

Compound 1 (trans-4-(Boc-amino)cyclohexanecarbaldehyde, 4.9 g) was dissolved in 17 mL of methanol, then an aqueous formaldehyde solution (4.21 g, with a concentration of 37% by mass) and an aqueous sodium hydroxide solution (6.5 mL, with a concentration of 45.3% by mass) were added dropwise. After the addition was completed, the mixture was warmed to 60° C. and stirred at 60° C. for 2 hours. After the reaction was finished, the reaction liquid was cooled to 25° C. and then evaporated to dryness under reduced pressure to obtain a crude product as a white solid. A small amount of water was added to the crude product for slurry. After filtration and drying, compound 2 was obtained as a white solid (4.8 g, yield 85.9%). ESI-MS(m/z)=260.2[M+H]⁺.

### (2.1.2) Synthesis of Compound 3

Compound 2 (4.8 g) obtained according to the preparation method in step (2.1.1) was dissolved in 25 mL of 1,4-dioxane, then a 1,4-dioxane solution of hydrochloric acid (25 mL, 4 M) was added. The mixture was stirred at 25° C. for 2 hours. After the reaction was completed, the reaction liquid was evaporated to dryness under reduced pressure to obtain compound 3 as a white solid (3.6 g, yield 99.4%).

### (2.1.3) Synthesis of Compound 11

Compound 3 (3.6 g) obtained according to the preparation method in step (2.1.2) was dissolved in 36 mL of DMF, then TEA (5.62 g), compound 10 (N-benzyloxycarbonyl-4-aminobutyric acid, 5.28 g) and HBTU (8.43 g) were added. The mixture was stirred at 25° C. for 16 hours. After the reaction was completed, the reaction liquid was added to 200 mL of saturated aqueous sodium bicarbonate solution. The mixture was extracted with 100 mL of ethyl acetate three times (3×100 mL). The organic phases were combined. The organic phase was washed once with 50 mL of saturated aqueous sodium chloride solution (1×50 mL), then dried over anhydrous sodium sulfate. The organic phase was evaporated to dryness under reduced pressure and purified by normal-phase column chromatography (eluent: dichloromethane/methanol=10/1, v/v) to obtain compound 11 as a white solid (2.3 g, yield 33.0%). ESI MS(m/z)=379.5[M+H]⁺.

### (2.1.4) Synthesis of Compound 12

Compound 11 (2.3 g) obtained according to the preparation method in step (2.1.3) was dissolved in 23 mL of methanol, then wet palladium on carbon (230 mg, with a loading of 10% by mass) was added. The reaction system was purged with hydrogen three times and then the mixture was stirred at 25° C. for 16 hours under a hydrogen atmosphere (15 psi). After the reaction was completed, the reaction liquid was filtered to obtain a filtrate. The filtrate was evaporated to dryness under reduced pressure to obtain compound 12 as a yellow oil (1.48 g, yield 99.8%).

### (2.1.5) Synthesis of Compound 13

Compound 12 (1.48 g) obtained according to the preparation method in step (2.1.4) was dissolved in 15 mL of DMF, then triethylamine (TEA, 1.22 g), compound 4 (1.35 g), and HBTU (3.45 g) were added. The mixture was stirred at 25° C. for 16 hours. After the reaction was completed, the reaction liquid was added to 150 mL of saturated aqueous sodium bicarbonate solution. The mixture was extracted with 50 mL of ethyl acetate three times (3×50 mL). The organic phases were combined. The organic phase was washed once with 30 mL of saturated aqueous sodium chloride solution (1×30 mL), then dried over anhydrous sodium sulfate. The organic phase was evaporated to dryness under reduced pressure and purified by reverse-phase column chromatography (C18 column, eluent: water/acetonitrile=5/1, v/v) to obtain compound 13 as a white solid (1.3 g, yield 31.8%). ESI-MS(m/z):674.3[M+H]⁺.

### (2.1.6) Synthesis of Compound 14

Compound 13 (1.1 g) obtained according to the preparation method in step (2.1.5) was dissolved in 11 mL of pyridine. The reaction system was cooled to 0° C. using an ice-water bath, and DMTrC1 (813 mg) was added in batches at 0° C. The reaction system was stirred at 0° C. for 1 hour. After the reaction was completed, methanol was added to the reaction liquid to quench the reaction. The solvent was evaporated, and the residue was purified by reverse-phase column chromatography (eluent: water/acetonitrile=1/4, v/v) to obtain compound 14 as a white solid (800 mg, yield 50.3%). ESI-MS(m/z):976.5[M+H]⁺.

### (2.1.7) Synthesis of Compound CR01013

At 25° C., compound 14 (550 mg) obtained according to the preparation method in step (2.1.6) was dissolved in 5 mL of dichloromethane (DCM), then 4,5-dicyanoimidazole (DCI, 53.2 mg) and compound 7 (2-cyanoethyl N,N,N′,N′-tetraisopropylphosphorodiamidite, 255.4 mg) were added. The reaction system was purged with nitrogen three times and then stirred at 25° C. for 1 hour under a nitrogen atmosphere. After the reaction was completed, the reaction liquid was washed twice with 5 mL of saturated aqueous sodium bicarbonate solution (2×5 mL), then washed once with 30 mL of saturated aqueous sodium chloride solution (1×30 mL). The organic phase was separated, dried over anhydrous sodium sulfate, and the solvent of the organic phase was evaporated under reduced pressure. The residue was purified by normal-phase column chromatography (eluent: dichloromethane/methanol=20/1, v/v) to obtain compound CR01013 as a white solid (532 mg, yield 80.4%). ESI-MS(m/z): 1176.7[M+H]⁺.

1H NMR (400 MHz, DMSO-d6) δ 0.95-1.05 (d, J=6.7 Hz, 5H), 1.06-1.15 (q, J=7.6 Hz, 8H), 1.15-1.21 (t, J=7.2 Hz, 14H), 1.72-1.80 (s, 3H), 1.84-1.92 (s, 3H), 1.94-2.07 (d, J=16.0 Hz, 7H), 2.07-2.14 (s, 3H), 2.64-2.72 (q, J=5.8 Hz, 2H), 2.74-2.89 (d, J=8.5 Hz, 2H), 3.35-3.56 (m, 4H), 3.57-3.70 (m, 4H), 3.71-3.77 (s, 6H), 3.81-3.93 (m, 1H), 3.96-4.09 (d, J=6.4 Hz, 3H), 6.82-6.97 (d, J=8.7 Hz, 4H), 7.17-7.27 (t, J=8.7 Hz, 5H), 7.27-7.34 (t, J=7.6 Hz, 2H), 7.34-7.43 (d, J=7.5 Hz, 2H).

### (2.2) Preparation of Compound CR01013Z

In the Preparation Example, the synthetic route for compound CR01013Z is as follows:

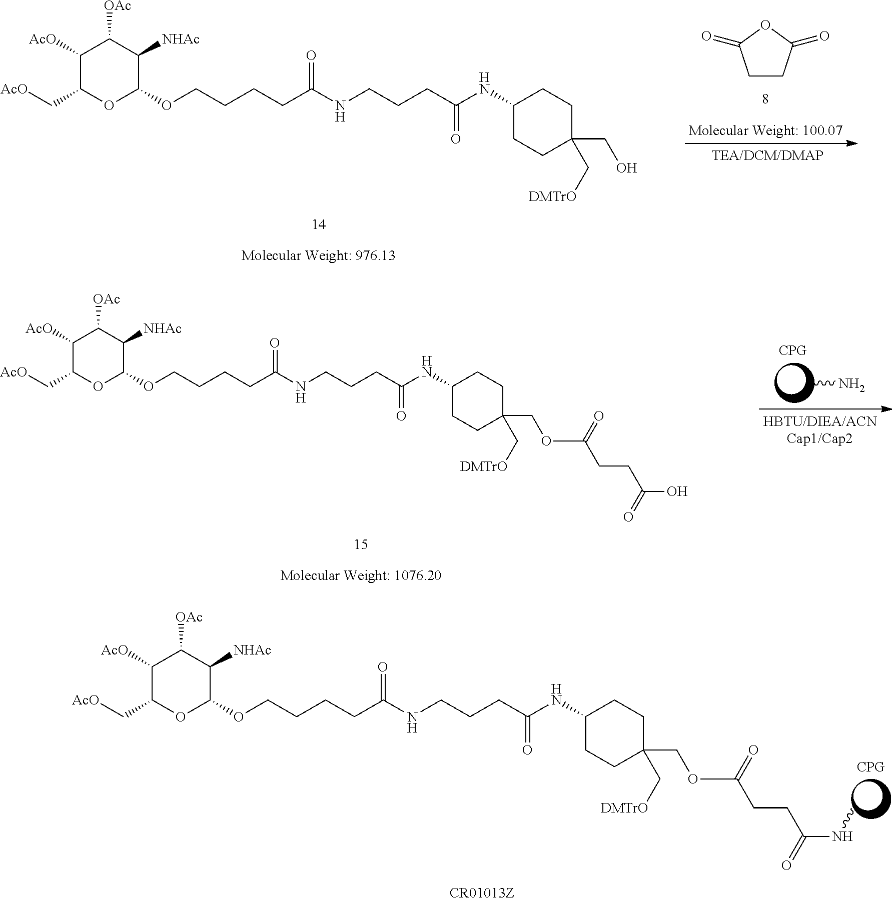

### (2.2.1) Synthesis of Compound 15

At 25° C., compound 14 (100 mg, 0.10 mmol) obtained according to the preparation method in step (2.1.6) was dissolved in 2 mL of dichloromethane, then triethylamine (25.9 mg, 0.25 mmol), DMAP (1.25 mg, 0.01 mmol) and compound 8 (succinic anhydride, 15.4 mg, 0.15 mmol) were added. The reaction system was stirred at 25° C. for 16 hours. After the reaction was completed, the solvent in the reaction liquid was evaporated, and the residue was purified by reverse-phase column chromatography (C18 column, eluent: water/acetonitrile=2/1, v/v) to obtain compound 15 as a yellow oil (110 mg, 0.10 mmol, yield 100%). ESI-MS(m/z)=1099.3[M+Na]⁺.

### (2.2.2) Synthesis of Compound CR01013Z

Compound 15 (50 mg, 0.04 mmol) obtained according to the preparation method in step (2.2.1) was dissolved in 10 mL of acetonitrile, then HBTU (24.2 mg, 0.06 mmol), DIEA (11.0 mg, 0.08 mmol) and aminoalkyl-CPG (1.06 g, loading capacity 80 μmol/g) were added. The reaction system was stirred at 25° C. for 16 hours. After the reaction was completed, the reaction liquid was filtered to obtain a filter cake. The filter cake was washed twice with 50 mL of dichloromethane (2×50 mL), three times with 50 mL of acetonitrile (3×50 mL) and once with 50 mL of ethyl acetate (1×50 mL) sequentially, and then dried under vacuum. Cap1 (4.8 mL), Cap2 (0.54 mL) and DMAP (2.59 mg) were added to the dried filter cake, and the reaction system was stirred at 25° C. for 5 hours. After the reaction was finished, the reaction liquid was filtered to obtain a filter cake. The filter cake was washed three times with 50 mL of acetonitrile (3×50 mL) and dried under vacuum to obtain compound CR01013Z (900 mg, loading capacity: 20-30 μmol/g).

Wherein, Cap1 and Cap2 are capping reagents. Cap1 is a pyridine/acetonitrile mixed solution comprising 20% N-methylimidazole by volume, with a volume ratio of pyridine to acetonitrile of 3:5. Cap2 is a 20% acetic anhydride solution in acetonitrile by volume.

### Preparation Example 3: Preparation of Double-Stranded Oligonucleotides(SiRNA)

### (3.1) Synthesis of the Sense Strand (SS)

The nucleoside monomers were sequentially linked in the 3′-to-5′ direction through the solid-phase phosphoramidite nucleic acid synthesis method. Each step of linking a nucleoside monomer involved four reactions: deprotection, coupling, capping, and oxidation or sulfurization. The synthesis conditions are as follows:
The nucleoside monomers were prepared into a 0.1M solution in acetonitrile.

The conditions for each deprotection reaction were the same. The conditions of the deprotection reaction: the temperature was 25° C.; the reaction time was 70 seconds; the deprotection reagent was a dichloromethane solution of dichloroacetic acid (3% by volume); the molar ratio of dichloroacetic acid to the 4,4′-dimethoxytrityl protecting group on the solid phase carrier was 5:1.

The conditions for each coupling reaction were the same. The conditions of the coupling reaction: the temperature was 25° C.; the molar ratio of the nucleic acid sequence attached to the solid-phase carrier to the nucleoside monomer was 1:10; the molar ratio of the nucleic acid sequence attached to the solid-phase carrier to the coupling reagent was 1:65; the reaction time was 600 seconds; the coupling reagent was an acetonitrile solution of 5-ethanethio-1H-tetrazolium at a concentration of 0.5M; the sulfuric acid reagent was a hydroxylated xanthate in a 0.2 M acetonitrile/pyridine mixed solution at a concentration of 0.2 M (volume ratio of acetonitrile to pyridine was 1:1).

The conditions for each capping reaction were the same. The conditions of the capping reaction: the temperature was 25° C.; the reaction time was 2 minutes; the capping reagent solution was a mixture of Cap1 and Cap2 with a molar ratio of 1:1; Cap1 was a pyridine/acetonitrile mixed solution comprising 20% (by volume) N-methylimidazole, with a volume ratio of pyridine to acetonitrile of 3:5; Cap2 was a 20% (by volume) acetic anhydride solution in acetonitrile; the molar ratio of N-methylimidazole in Cap1, acetic anhydride in Cap2, and the nucleic acid sequence linked on the solid-phase carrier was 1:1:1.

The conditions for each oxidation reaction were the same. The conditions for the oxidation reaction: the temperature was 25° C.; the reaction time was 3 seconds; the oxidizing reagent was an iodine solution with a concentration of 0.05 M, and the molar ratio of iodine to the nucleic acid sequence linked on the solid-phase carrier in the coupling reaction was 30:1; the oxidation reaction was conducted in a water/pyridine mixed solvent (volume ratio of water to pyridine was 1:9). The conditions for the sulfation reaction: the temperature was 25° C.; the reaction time was 360 seconds; the thio-reagent was a 0.2M solution of hydrogen xanthate in pyridine, and the molar ratio of the thio-reagent to the nucleic acid sequence linked on the solid-phase carrier in the coupling reaction was 4:1; the sulfurization reaction was conducted in a water/pyridine mixed solvent (volume ratio of water to pyridine was 1:9).

After the linking of the last nucleoside monomer was finished, the nucleic acid sequence linked on the solid-phase support was sequentially subjected to cleavage, deprotection, purification, and desalination, and then freeze-dried to obtain the sense strand, wherein:
The conditions for cleavage and deprotection were as follows: the synthesized nucleotide sequence linked to the solid-phase carrier was added to a 25% ammonia solution by mass at a dosage of 0.5 mL/μmol. The mixture was reacted at 55° C. for 16 hours. Then the solvent was removed, and the residue was concentrated to dryness under vacuum. After the ammonia treatment, relative to the amount of single-stranded nucleic acid, the product was dissolved in 0.4 mL/μmol of N-methylpyrrolidone. Subsequently, 0.3 mL/μmol of triethylamine and 0.6 mL/μmol of triethylamine trihydrofluoride were added to remove the 2′-O-TBDMS protection group on the ribose.

The conditions for purification and desalination: the nucleic acid was purified by gradient elution with NaCl using a preparative ion-exchange chromatography column (Source 15Q). Specifically, Eluent 1 was a 20 mM sodium phosphate solution (pH=8.1) in a water/acetonitrile mixed solvent (volume ratio of water to acetonitrile was 9:1). Eluent 2 was a solution comprising 1.5 M sodium chloride and 20 mM sodium phosphate (pH=8.1) in a water/acetonitrile mixed solvent (volume ratio of water to acetonitrile was 9:1). The elution gradient was from Eluent 1: Eluent=(100:0)-(50:50). After collecting and combining the product eluates, desalination was carried out using a reverse-phase chromatography purification column. The desalination conditions involved using a Sephadex column packed with Sephadex G25 and eluting with deionized water.

Detection: purity was detected using ion exchange chromatography (IEX-HPLC); molecular weight was detected using liquid chromatography-mass spectrometry (LC-MS); by comparing the measured molecular weight with the theoretical value, if they were consistent, it indicated that the sense strand of siRNA was obtained.

### (3.2) Synthesis of the Antisense Strand (AS)

The antisense strand was synthesized using a solid-phase carrier. The conditions for deprotection, coupling, capping, oxidation or sulfurization reactions, cleavage, deprotection, purification, and desalination in the solid-phase synthesis method of the antisense strand were the same as those for synthesizing the sense strand in step (3.1).

Detection: purity was detected using ion exchange chromatography (IEX-HPLC); molecular weight was detected using liquid chromatography-mass spectrometry (LC-MS); by comparing the measured molecular weight with the theoretical value, if they were consistent, it indicated that the antisense strand of siRNA was obtained.

(3.3) Synthesis of the siRNA Duplex

The sense strand synthesized in step (3.1) and the antisense strand synthesized in step (3.2) were mixed in an equimolar ratio. The mixture was dissolved in water for injection, heated to 95° C., slowly cooled to room temperature, and then maintained at room temperature for 10 minutes. Through this process, the sense strand and the antisense strand formed a double-stranded structure via hydrogen bonds, thus obtaining the siRNA with the sense strand and antisense strand shown in Table 1.

According to the method provided by the present disclosure, the unmodified double-stranded oligonucleotides (siRNA) shown in Table 4 were obtained.

TABLE 4
Unmodified siRNA

| siRNA | Sense strand | SEQ | Antisense strand | SEQ |
|---|---|---|---|---|
| Number | (5′-3′) | ID | (5′-3′) | ID |
| 1 | CAUCCACAAUGAG | No. 1 | GGUACUCUCAUUG | No. 2 |
| | AGUACC | | UGGAUGAC | |
| 2 | UCAACUGGAUGAA | No. 3 | AGUUUCUUCAUCC | No. 4 |
| | GAAACU | | AGUUGAGG | |
| 3 | CCUGUUUGCUGUG | No. 5 | AUCAUACACAGCA | No. 6 |
| | UAUGAU | | AACAGGAA | |
| 4 | CAAGUUGAGAACA | No. 7 | AAUUUUUGUUCUC | No. 8 |
| | AAAAUU | | AACUUGAA | |
| 5 | GUUUUAAAAUUAA | No. 9 | UAUACUUUAAUUU | No. 10 |
| | AGUAUA | | UAAAACCC | |
| 6 | AAGUAUACAUUUU | No. 11 | AAUGCAAAAAUGU | No. 12 |
| | UGCAUU | | AUACUUUA | |

When the sequence listing production software is used to generate RNA sequences, it requires representing U as T. Therefore, the siRNA sequences of the present disclosure cannot be accurately represented in the sequence listing. All sequences shall be subject to those recited in the description.

According to the method provided by the present disclosure, the modified double-stranded oligonucleotides (siRNA) shown in Table 5 were obtained.

TABLE 5
Sequence information of the modified siRNA

| Group | Sense strand (5′-3′) | Antisense strand (5′-3′) |
|---|---|---|
| 1 | CmsAmsAmGmUmUmGfAfGfA | AmsAfsUmUmUmUfUmGmUmUf |
| | fAmC(moe)AmAmAmAmAmUm | CmUmCmAfA(moe)CfUmUmGms |
| | Um | AmsAm |
| 2 | CmsAmsAmGmUmUmGfAfGfA | AmsAfsUmUmUmUfUmGmUmU |
| | fAmC(moe)AmAmAmAmAmUm | mCfUmCmAfA(moe)CfUmUmGm |
| | Um | sAmsAm |
| 3 | CmsAmsAmGmUmUmGfAfGfA | AmsAfsUmUmUmUfUmGmUmUf |
| | fAmCmAmAmAmAmAmT(moe) | CmUmCmAfA(moe)CfUmUmGms |
| | Um | AmsAm |
| 4 | CmsAmsAmGmUmUmGfAfGfA | AmsAfsUmUmUmUfUmGmUmUf |
| | fAmC(moe)AmAmAmAmAmT(moe) | CmUmCmAfA(moe)CfUmUmGms |
| | Um | AmsAm |
| 5 | CmsAmsAmGmUmUmGfAfGfA | AmsAfsUmUmUmUfUmG(moe)U |
| | fAmCmAmAmAmAmAmT(moe) | mUfCmUmCmAfAmCfUmUmGms |
| | Um | AmsAm |
| 6 | GmsUmsUmUmUmAmAfAfAfU | UmsAfsUmAmCmUfUmUmAmAf |
| | fUmA(moe)AmAmGmUmAmU | UmUmUmUfA(moe)AfAmAmCms |
| | mAm | CmsCm |
| 7 | GmsUmsUmUmUmAmAfAfAfU | UmsAfsUmAmCmUfUmUmAmA |
| | fUmA(moe)AmAmGmUmAmU | mUfUmUmUfA(moe)AfAmAmCm |
| | mAm | sCmsCm |
| 8 | GmsUmsUmUmUmAmAfAfAfU | UmsAfsUmAmCmUfUmUmAmAf |
| | fUmAmAmAmGmUmAmT(moe) | UmUmUmUfA(moe)AfAmAmCms |
| | Am | CmsCm |
| 9 | GmsUmsUmUmUmAmAfAfAfU | UmsAfsUmAmCmUfUmUmAmAf |
| | fUmA(moe)AmAmGmUmAmT(moe) | UmUmUmUfA(moe)AfAmAmCms |
| | Am | CmsCm |
| 10 | GmsUmsUmUmUmAmAfAfAfU | UmsAfsUmAmCmUfUmT(moe)A |
| | fUmAmAmAmGmUmAmT(moe) | mAfUmUmUmUfAmAfAmAmCm |
| | Am | sCmsCm |
| 11 | AmsAmsGmUmAmUmAfCfAfU | AmsAfsUmGmCmAfAmAmAmA |
| | fUmT(moe)UmUmGmCmAmUm | mUmGmUmAfT(moe)AfCmUmU |
| | Um | msUmsAm |
| 12 | AmsAmsGmUmAmUmAfCfAfU | AmsAfsUmGmCmAfAmAmAmAf |
| | fUmT(moe)UmUmGmCmAmUm | UmGmUmAfT(moe)AfCmUmUms |
| | Um | UmsAm |
| 13 | AmsAmsGmUmAmUmAfCfAfU | AmsAfsUmGmCmAfAmAmAmA |
| | fUmUmUmUmGmCmAmT(moe) | mUfGmUmAfT(moe)AfCmUmUm |
| | Um | sUmsAm |
| 14 | AmsAmsGmUmAmUmAfCfAfU | AmsAfsUmGmCmAfAmAmAmAf |
| | fUmT(moe)UmUmGmCmAmT(moe) | UmGmUmAfT(moe)AfCmUmUms |
| | Um | UmsAm |
| 15 | AmsAmsGmUmAmUmAfCfAfU | AmsAfsUmGmCmAfAmAmAmAf |
| | fUmUmUmUmGmCmAmT(moe) | UmGmUmAfT(moe)AfCmUmUms |
| | Um | UmsAm |
| 16 | CmsAmsAmGmUmUmGfAfGfA | AmsAfsUmUmUmUfUmGmUmUf |
| | fAmCmAmAmAmAmAmUmUm | CmUmCmAfA(moe)CfUmUmGms |
| | | AmsAm |
| 17 | GmsUmsUmUmUmAmAfAfAfU | UmsAfsUmAmCmUfUmUmAmA |
| | fUmAmAmAmGmUmAmUmAm | mUfUmUmUfA(moe)AfAmAmCm |
| | | sCmsCm |
| 18 | AmsAmsGmUmAmUmAfCfAfU | AmsAfsUmGmCmAfAmAmAmAf |
| | fUmUmUmUmGmCmAmUmUm | UmGmUmAfUmAfCmUmUmsUm |
| | | sAm |
| 19 | AmsAmsGmUmAmUmAfCfAfU | AmsAfsUmGmCmAfAmAmAmAf |
| | fUmUmUmUmGmCmAmUmUm | UmGmUmAfT(moe)AfCmUmUms |
| | | UmsAm |
| 20 | GmsUmsUmUmUmAmAfAfAfU | UmsAfsUmAmCmUfUmUmAfAm |
| | fUmAmAmAmGmUmAmUmAm | UmUmUmUfA(moe)AfAmAmCms |
| | | CmsCm |
| 21 | GmsUmsUmUmUmAmAfAfAfU | UmsAfsUmAmCmUfUmUmAmA |
| | fUmAmAmAmGmUmAmUmAm | mUmUfUmUfA(moe)AfAmAmCm |
| | | sCmsCm |
| 22 | AmsAmsGmUmAmUmAfCfAfU | AmsAfsUmGmCmAfAmAmAfAm |
| | fUmUmUmUmGmCmAmUmUm | UmGmUmAfT(moe)AfCmUmUms |
| | | UmsAm |
| 23 | AmsAmsGmUmAmUmAfCfAfU | AmsAfsUmGmCmAfAmAmAmA |
| | fUmUmUmUmGmCmAmUmUm | mUmGfUmAfT(moe)AfCmUmUm |
| | | sUmsAm |
| 24 | AmsAmsGmUmAmUmAfCfAfU | AmsAfsUmGmCmAfAmAmAfAm |
| | fUmUmUmUmGmCmAmUmUm | UmGmUmAfU(moe)AfCmUmUms |
| | | UmsAm |
| 25 | AmsAmsGmUmAmUmAfCfAfU | AmsAfsUmGmCmAfAmAmAmA |
| | fUmUmUmUmGmCmAmUmUm | mUmGfUmAfU(moe)AfCmUmUm |
| | | sUmsAm |
| 26 | CmsAmsAmGmUmUmGfAfGfA | AmsAfsUmUmUmUfUmGmUmU |
| | fAmCmAmAmAmAmAmUmUm | mCmUfCmAfA(moe)CfUmUmGm |
| | | sAmsAm |
| 27 | CmsAmsAmGmUmUmGfAfGfA | AmsAfsUmUmUmUfUmGmUfUm |
| | fAmCmAmAmAmAmAmUmUm | CmUmCmAfA(moe)CfUmUmGms |
| | | AmsAm |
| 28 | CmsAmsAmGmUmUmGfAfGfA | AmsAfsUmUmUmUfUmGmUmU |
| | fAmCmAmAmAmAmAmUmUm | mCmUfCmAfAmCfUmUmGmsAm |
| | | sAm |
| 29 | CmsAmsAmGmUmUmGfAfGfA | AmsAfsUmUmUmUfUmGmUfUm |
| | fAmCmAmAmAmAmAmUmUm | CmUmCmAfAmCfUmUmGmsAm |
| | | sAm |
| 30 | GmsUmsUmUmUmAmAfAfAfU | UmsAfsUmAmCmUfUmUmAfAm |
| | fUmAmAmAmGmUmAmUmAm | UmUmUmUfAmAfAmAmCmsCm |
| | | sCm |
| 31 | GmsUmsUmUmUmAmAfAfAfU | UmsAfsUmAmCmUfUmUmAmA |
| | fUmAmAmAmGmUmAmUmAm | mUmUfUmUfAmAfAmAmCmsC |
| | | msCm |
| 32 | AmsAmsGmUmAmUmAfCfAfU | AmsAfsUmGmCmAfAmAmAfAm |
| | fUmUmUmUmGmCmAmUmUm | UmGmUmAfUmAfCmUmUmsUm |
| | | sAm |
| 33 | AmsAmsGmUmAmUmAfCfAfU | AmsAfsUmGmCmAfAmAmAmA |
| | fUmUmUmUmGmCmAmUmUm | mUmGfUmAfUmAfCmUmUmsU |
| | | msAm |
| 34 | CmsAmsUmCmCmAmCfAfAfUf | GmsGfsUmAmCmUfCmUmCfAm |
| | GmAmGmAmGmUmAmCmCm | UmUmGmUfGmGfAmUmGmsAm |
| | | sCm |
| 35 | CmsAmsUmCmCmAmCfAfAfUf | GmsGfsUmAmCmUfCmUmCmA |
| | GmAmGmAmGmUmAmCmCm | mUmUfGmUfGmGfAmUmGmsA |
| | | msCm |
| 36 | CmsAmsUmCmCmAmCfAfAfUf | GmsGfsUmAmCmUfCmUmCfAm |
| | GmAmGmAmGmUmAmCmCm | UmUmGmUfG(moe)GfAmUmGms |
| | | AmsCm |
| 37 | CmsAmsUmCmCmAmCfAfAfUf | GmsGfsUmAmCmUfCmUmCmA |
| | GmAmGmAmGmUmAmCmCm | mUmUfGmUfG(moe)GfAmUmGm |
| | | sAmsCm |
| 38 | UmsCmsAmAmCmUmGfGfAfUf | AmsGfsUmUmUmCfUmUmCfAm |
| | GmAmAmGmAmAmAmCmUm | UmCmCmAfGmUfUmGmAmsGm |
| | | sGm |
| 39 | UmsCmsAmAmCmUmGfGfAfUf | AmsGfsUmUmUmCfUmUmCmA |
| | GmAmAmGmAmAmAmCmUm | mUmCfCmAfGmUfUmGmAmsG |
| | | msGm |
| 40 | UmsCmsAmAmCmUmGfGfAfUf | AmsGfsUmUmUmCfUmUmCfAm |
| | GmAmAmGmAmAmAmCmUm | UmCmCmAfG(moe)UfUmGmAms |
| | | GmsGm |
| 41 | UmsCmsAmAmCmUmGfGfAfUf | AmsGfsUmUmUmCfUmUmCmA |
| | GmAmAmGmAmAmAmCmUm | mUmCfCmAfG(moe)UfUmGmAm |
| | | sGmsGm |
| 42 | CmsCmsUmGmUmUmUfGfCfUf | AmsUfsCmAmUmAfCmAmCfAm |
| | GmUmGmUmAmUmGmAmUm | GmCmAmAfAmCfAmGmGmsAm |
| | | sAm |
| 43 | CmsCmsUmGmUmUmUfGfCfUf | AmsUfsCmAmUmAfCmAmCmA |
| | GmUmGmUmAmUmGmAmUm | mGmCfAmAfAmCfAmGmGmsA |
| | | msAm |
| 44 | CmsCmsUmGmUmUmUfGfCfUf | AmsUfsCmAmUmAfCmAmCfAm |
| | GmUmGmUmAmUmGmAmUm | GmCmAmAfA(moe)CfAmGmGms |
| | | AmsAm |
| 45 | CmsCmsUmGmUmUmUfGfCfUf | AmsUfsCmAmUmAfCmAmCmA |
| | GmUmGmUmAmUmGmAmUm | mGmCfAmAfA(moe)CfAmGmGm |
| | | sAmsAm |

### Preparation Example 4: Synthesis of the Compound with the (CR01008×3) Carrier Conjugated to the 3′-End of the Sense Strand

### (4.1) Synthesis of Sense Strand

Through the solid-phase phosphoramidite nucleic acid synthesis method, starting from the above compound CR01008Z that linked to the solid-phase carrier, nucleoside monomers were cyclically and sequentially connected one by one in the 3′-5′ direction according to the nucleotide sequence (the compound CR01008 was regarded as a nucleoside monomer).

Each connection of a nucleoside monomer involves four steps of reactions: deprotection, coupling, capping, and oxidation or sulfurization. The conditions for deprotection, coupling, capping, oxidation or sulfurization reactions, cleavage, deprotection, purification, and desalination in the synthesis of the sense strand in the Preparation Example are the same as those in the synthesis of the sense strand in step (3.1) of Preparation Example 3.

In the step, during the synthesis procedure of the sense strand, three clusters of CR01008 were synthesized, denoted as (CR01008)×3 or (CR01008×3).

The structural Formula of the three clusters of CR01008 is as follows:

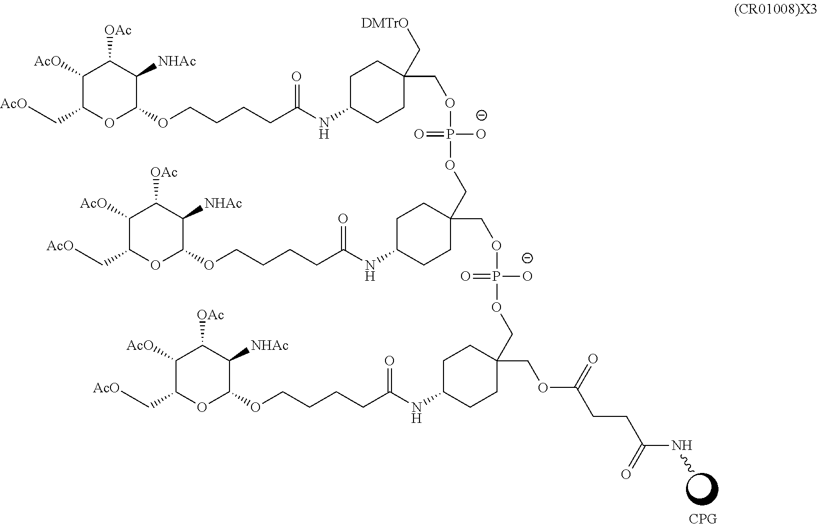

### (5.2) Synthesis of the Antisense Strand:

The antisense strand of the Preparation Example was synthesized according to the antisense strand synthesis method shown in step (3.2) in Preparation Example 3.

(5.3) Synthesis of siRNA Conjugates

The oligonucleotide-conjugated compound of the Preparation Example was synthesized according to the method shown in step (3.3) in Preparation Example 3.

Wherein, when the ligand is three clusters of CR01008, the structural formula of the siRNA conjugate is as follows:

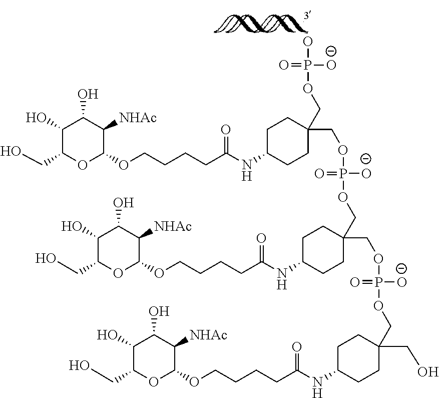

Wherein, [symbol] represents siRNA. The (CR01008×3) carrier is [symbol] conjugated to the 3′-end of the sense strand of the siRNA. The oligonucleotide-conjugated compound shown in Table 6 was obtained according to the method provided by the present disclosure.

TABLE 6
Sequence information of the oligonucleotide-conjugated compound

| Compound | | |
|---|---|---|
| number | Sense strand (5′-3′) | Antisense strand (5′-3′) |
| RZ003028 | CmsAmsAmGmUmUmGfAf | AmsAfsUmUmUmUfUmGmU |
| | GfAfAmC(moe)AmAmAmA | mUfCmUmCmAfA(moe)CfU |
| | mAmUmUm_(CR01008 × 3) | mUmGmsAmsAm |
| RZ003029 | CmsAmsAmGmUmUmGfAf | AmsAfsUmUmUmUfUmGmU |
| | GfAfAmC(moe)AmAmAmA | mUmCfUmCmAfA(moe)CfU |
| | mAmUmUm_(CR01008 × 3) | mUmGmsAmsAm |
| RZ003030 | CmsAmsAmGmUmUmGfAf | AmsAfsUmUmUmUfUmGmU |
| | GfAfAmCmAmAmAmAmA | mUfCmUmCmAfA(moe)CfU |
| | mT(moe)Um_(CR01008 × 3) | mUmGmsAmsAm |
| RZ003031 | CmsAmsAmGmUmUmGfAf | AmsAfsUmUmUmUfUmGmU |
| | GfAfAmC(moe)AmAmAmA | mUfCmUmCmAfA(moe)CfU |
| | mAmT(moe)Um_(CR01008 × | mUmGmsAmsAm |
| | 3) | |
| RZ003032 | CmsAmsAmGmUmUmGfAf | AmsAfsUmUmUmUfUmG(m |
| | GfAfAmCmAmAmAmAmA | oe)UmUfCmUmCmAfAmCfU |
| | mT(moe)Um_(CR01008 × 3) | mUmGmsAmsAm |
| RZ003041 | GmsUmsUmUmUmAmAfAf | UmsAfsUmAmCmUfUmUmA |
| | AfUfUmA(moe)AmAmGmU | mAfUmUmUmUfA(moe)AfA |
| | mAmUmAm_(CR01008 × 3) | mAmCmsCmsCm |
| RZ003042 | GmsUmsUmUmUmAmAfAf | UmsAfsUmAmCmUfUmUmA |
| | AfUfUmA(moe)AmAmGmU | mAmUfUmUmUfA(moe)AfA |
| | mAmUmAm_(CR01008 × 3) | mAmCmsCmsCm |
| RZ003043 | GmsUmsUmUmUmAmAfAf | UmsAfsUmAmCmUfUmUmA |
| | AfUfUmAmAmAmGmUmA | mAfUmUmUmUfA(moe)AfA |
| | mT(moe)Am_(CR01008 × 3) | mAmCmsCmsCm |
| RZ003044 | GmsUmsUmUmUmAmAfAf | UmsAfsUmAmCmUfUmUmA |
| | AfUfUmA(moe)AmAmGmU | mAfUmUmUmUfA(moe)AfA |
| | mAmT(moe)Am_(CR01008 × | mAmCmsCmsCm |
| | 3) | |
| RZ003045 | GmsUmsUmUmUmAmAfAf | UmsAfsUmAmCmUfUmT(mo |
| | AfUfUmAmAmAmGmUmA | e)AmAfUmUmUmUfAmAfA |
| | mT(moe)Am_(CR01008 × 3) | mAmCmsCmsCm |
| RZ003051 | AmsAmsGmUmAmUmAfCf | AmsAfsUmGmCmAfAmAmA |
| | AfUfUmT(moe)UmUmGmC | mAmUmGmUmAfT(moe)AfC |
| | mAmUmUm_(CR01008 × 3) | mUmUmsUmsAm |
| RZ003052 | AmsAmsGmUmAmUmAfCf | AmsAfsUmGmCmAfAmAmA |
| | AfUfUmT(moe)UmUmGmC | mAfUmGmUmAfT(moe)AfC |
| | mAmUmUm_(CR01008 × 3) | mUmUmsUmsAm |
| RZ003053 | AmsAmsGmUmAmUmAfCf | AmsAfsUmGmCmAfAmAmA |
| | AfUfUmUmUmUmGmCmA | mAmUfGmUmAfT(moe)AfC |
| | mT(moe)Um_(CR01008 × 3) | mUmUmsUmsAm |
| RZ003054 | AmsAmsGmUmAmUmAfCf | AmsAfsUmGmCmAfAmAmA |
| | AfUfUmT(moe)UmUmGmC | mAfUmGmUmAfT(moe)AfC |
| | mAmT(moe)Um_(CR01008 × | mUmUmsUmsAm |
| | 3) | |
| RZ003055 | AmsAmsGmUmAmUmAfCf | AmsAfsUmGmCmAfAmAmA |
| | AfUfUmUmUmUmGmCmA | mAfUmGmUmAfT(moe)AfC |
| | mT(moe)Um_(CR01008 × 3) | mUmUmsUmsAm |
| RZ003062 | CmsAmsAmGmUmUmGfAf | AmsAfsUmUmUmUfUmGmU |
| | GfAfAmCmAmAmAmAmA | mUfCmUmCmAfA(moe)CfU |
| | mUmUm_(CR01008 × 3) | mUmGmsAmsAm |
| RZ003064 | GmsUmsUmUmUmAmAfAf | UmsAfsUmAmCmUfUmUmA |
| | AfUfUmAmAmAmGmUmA | mAmUfUmUmUfA(moe)AfA |
| | mUmAm_(CR01008 × 3) | mAmCmsCmsCm |
| RZ003065 | AmsAmsGmUmAmUmAfCf | AmsAfsUmGmCmAfAmAmA |
| | AfUfUmUmUmUmGmCmA | mAfUmGmUmAfUmAfCmU |
| | mUmUm_(CR01008 × 3) | mUmsUmsAm |
| RZ003066 | AmsAmsGmUmAmUmAfCf | AmsAfsUmGmCmAfAmAmA |
| | AfUfUmUmUmUmGmCmA | mAfUmGmUmAfT(moe)AfC |
| | mUmUm_(CR01008 × 3) | mUmUmsUmsAm |
| RZ003069 | GmsUmsUmUmUmAmAfAf | UmsAfsUmAmCmUfUmUmA |
| | AfUfUmAmAmAmGmUmA | fAmUmUmUmUfA(moe)AfA |
| | mUmAm_(CR01008 × 3) | mAmCmsCmsCm |
| RZ003070 | GmsUmsUmUmUmAmAfAf | UmsAfsUmAmCmUfUmUmA |
| | AfUfUmAmAmAmGmUmA | mAmUmUfUmUfA(moe)AfA |
| | mUmAm_(CR01008 × 3) | mAmCmsCmsCm |
| RZ003071 | AmsAmsGmUmAmUmAfCf | AmsAfsUmGmCmAfAmAmA |
| | AfUfUmUmUmUmGmCmA | fAmUmGmUmAfT(moe)AfC |
| | mUmUm_(CR01008 × 3) | mUmUmsUmsAm |
| RZ003072 | AmsAmsGmUmAmUmAfCf | AmsAfsUmGmCmAfAmAmA |
| | AfUfUmUmUmUmGmCmA | mAmUmGfUmAfT(moe)AfC |
| | mUmUm_(CR01008 × 3) | mUmUmsUmsAm |
| RZ003073 | AmsAmsGmUmAmUmAfCf | AmsAfsUmGmCmAfAmAmA |
| | AfUfUmUmUmUmGmCmA | fAmUmGmUmAfU(moe)AfC |
| | mUmUm_(CR01008 × 3) | mUmUmsUmsAm |
| RZ003074 | AmsAmsGmUmAmUmAfCf | AmsAfsUmGmCmAfAmAmA |
| | AfUfUmUmUmUmGmCmA | mAmUmGfUmAfU(moe)AfC |
| | mUmUm_(CR01008 × 3) | mUmUmsUmsAm |
| RZ003075 | CmsAmsAmGmUmUmGfAf | AmsAfsUmUmUmUfUmGmU |
| | GfAfAmCmAmAmAmAmA | mUmCmUfCmAfA(moe)CfU |
| | mUmUm_(CR01008 × 3) | mUmGmsAmsAm |
| RZ003076 | CmsAmsAmGmUmUmGfAf | AmsAfsUmUmUmUfUmGmU |
| | GfAfAmCmAmAmAmAmA | fUmCmUmCmAfA(moe)CfU |
| | mUmUm_(CR01008 × 3) | mUmGmsAmsAm |
| RZ003077 | CmsAmsAmGmUmUmGfAf | AmsAfsUmUmUmUfUmGmU |
| | GfAfAmCmAmAmAmAmA | mUmCmUfCmAfAmCfUmU |
| | mUmUm_(CR01008 × 3) | mGmsAmsAm |
| RZ003078 | CmsAmsAmGmUmUmGfAf | AmsAfsUmUmUmUfUmGmU |
| | GfAfAmCmAmAmAmAmA | fUmCmUmCmAfAmCfUmU |
| | mUmUm_(CR01008 × 3) | mGmsAmsAm |
| RZ003079 | GmsUmsUmUmUmAmAfAf | UmsAfsUmAmCmUfUmUmA |
| | AfUfUmAmAmAmGmUmA | fAmUmUmUmUfAmAfAmA |
| | mUmAm_(CR01008 × 3) | mCmsCmsCm |
| RZ003080 | GmsUmsUmUmUmAmAfAf | UmsAfsUmAmCmUfUmUmA |
| | AfUfUmAmAmAmGmUmA | mAmUmUfUmUfAmAfAmA |
| | mUmAm_(CR01008 × 3) | mCmsCmsCm |
| RZ003081 | AmsAmsGmUmAmUmAfCf | AmsAfsUmGmCmAfAmAmA |
| | AfUfUmUmUmUmGmCmA | fAmUmGmUmAfUmAfCmU |
| | mUmUm_(CR01008 × 3) | mUmsUmsAm |
| RZ003082 | AmsAmsGmUmAmUmAfCf | AmsAfsUmGmCmAfAmAmA |
| | AfUfUmUmUmUmGmCmA | mAmUmGfUmAfUmAfCmU |
| | mUmUm_(CR01008 × 3) | mUmsUmsAm |
| RZ003083 | CmsAmsUmCmCmAmCfAfA | GmsGfsUmAmCmUfCmUmC |
| | fUfGmAmGmAmGmUmAm | fAmUmUmGmUfGmGfAmU |
| | CmCm_(CR01008 × 3) | mGmsAmsCm |
| RZ003084 | CmsAmsUmCmCmAmCfAfA | GmsGfsUmAmCmUfCmUmC |
| | fUfGmAmGmAmGmUmAm | mAmUmUfGmUfGmGfAmU |
| | CmCm_(CR01008 × 3) | mGmsAmsCm |
| RZ003085 | CmsAmsUmCmCmAmCfAfA | GmsGfsUmAmCmUfCmUmC |
| | fUfGmAmGmAmGmUmAm | fAmUmUmGmUfG(moe)GfA |
| | CmCm_(CR01008 × 3) | mUmGmsAmsCm |
| RZ003086 | CmsAmsUmCmCmAmCfAfA | GmsGfsUmAmCmUfCmUmC |
| | fUfGmAmGmAmGmUmAm | mAmUmUfGmUfG(moe)GfA |
| | CmCm_(CR01008 × 3) | mUmGmsAmsCm |
| RZ003087 | UmsCmsAmAmCmUmGfGf | AmsGfsUmUmUmCfUmUmC |
| | AfUfGmAmAmGmAmAmA | fAmUmCmCmAfGmUfUmG |
| | mCmUm_(CR01008 × 3) | mAmsGmsGm |
| RZ003088 | UmsCmsAmAmCmUmGfGf | AmsGfsUmUmUmCfUmUmC |
| | AfUfGmAmAmGmAmAmA | mAmUmCfCmAfGmUfUmG |
| | mCmUm_(CR01008 × 3) | mAmsGmsGm |
| RZ003089 | UmsCmsAmAmCmUmGfGf | AmsGfsUmUmUmCfUmUmC |
| | AfUfGmAmAmGmAmAmA | fAmUmCmCmAfG(moe) UfU |
| | mCmUm_(CR01008 × 3) | mGmAmsGmsGm |
| RZ003090 | UmsCmsAmAmCmUmGfGf | AmsGfsUmUmUmCfUmUmC |
| | AfUfGmAmAmGmAmAmA | mAmUmCfCmAfG(moe)UfU |
| | mCmUm_(CR01008 × 3) | mGmAmsGmsGm |
| RZ003091 | CmsCmsUmGmUmUmUfGf | AmsUfsCmAmUmAfCmAmC |
| | CfUfGmUmGmUmAmUmG | fAmGmCmAmAfAmCfAmG |
| | mAmUm_(CR01008 × 3) | mGmsAmsAm |
| RZ003092 | CmsCmsUmGmUmUmUfGf | AmsUfsCmAmUmAfCmAmC |
| | CfUfGmUmGmUmAmUmG | mAmGmCfAmAfAmCfAmG |
| | mAmUm_(CR01008 × 3) | mGmsAmsAm |
| RZ003093 | CmsCmsUmGmUmUmUfGf | AmsUfsCmAmUmAfCmAmC |
| | CfUfGmUmGmUmAmUmG | fAmGmCmAmAfA(moe)CfA |
| | mAmUm_(CR01008 × 3) | mGmGmsAmsAm |
| RZ003094 | CmsCmsUmGmUmUmUfGf | AmsUfsCmAmUmAfCmAmC |
| | CfUfGmUmGmUmAmUmG | mAmGmCfAmAfA(moe)CfA |
| | mAmUm_(CR01008 × 3) | mGmGmsAmsAm |

“_(CR01008 × 3)”(CR01008 × 3) indicates that the ligand conjugate is connected to the 3′-end of the sense strand.

Unless otherwise specified, the meanings of base composition and modifications according to each example of the present disclosure are as follows: The capital letters A, U, G, C, and T represent the base composition of nucleotides respectively. The lowercase letter m indicates that the nucleotide adjacent to the left of the letter m is a nucleotide modified with 2′-O-methyl (also referred to: 2′-methoxy modification). The lowercase letter f indicates that the nucleotide adjacent to the left of the letter f is a nucleotide modified with 2′-fluoro. The lowercase letter d indicates that the nucleotide adjacent to the left of the letter d is a ribonucleic acid modified with 2′-deoxy (also referred to: deoxyribonucleic acid). (moe) indicates that the nucleotide adjacent to the left of the combination identifier (moe) is a nucleotide modified with 2′-O-methoxyethyl. The lowercase letter s indicates that the two nucleotides adjacent to the left and right of the letter s are connected by a phosphorothioate bond.

### Biological Detection Experiments

In the present disclosure, unless otherwise specified, all siRNA sequences used in the present disclosure were commissioned to be synthesized by Kunshan Aotai Biotechnology Co., Ltd. All PCR primers used in the present disclosure were commissioned to be synthesized by Sangon Biotech (Shanghai) Co., Ltd. The human hepatocellular carcinoma cell line Huh7 used in the present disclosure was purchased from Wuhan Pricella Biotechnology Co., Ltd. The experimental animals, C57BL/6J mice, used in the present disclosure were purchased from Zhejiang Vital River Laboratory Animal Technology Co., Ltd. The hREN×hAGT transgenic mice used in the present disclosure were purchased from Cyagen (Suzhou) Biosciences Inc. The SHR rats used in the present disclosure were purchased from Beijing Vital River Laboratory Animal Technology Co., Ltd. The blood pressure of mice and rats in the present disclosure was measured using a full-set intelligent non-invasive blood pressure system. The serum samples of mice and rats in the present disclosure were detected using a human angiotensinogen kit.

In the present disclosure, unless otherwise specified, the real-time PCR detection data of in-vivo activity experiments involved in the present disclosure are all calculated by the ΔΔCt method for the relative quantification of the target gene mRNA in each test group. The calculation method is summarized as follows:

$$Δ Ct ( test group ) = Ct ( target gene in the test group ) - Ct ( reference gene in the test group )$$

$$Δ Ct ( control group ) = Ct ( target gene in the control group ) - Ct ( reference gene in the control group )$$

$$Δ Δ Ct ( test group ) = Δ Ct ( test group ) - Δ Ct ( average of the control group )$$

$$Δ Δ Ct ( control group ) = Δ Ct ( control group ) - Δ Ct ( average of the control group )$$

Using the control group as a reference, the mRNA expression level of the target gene in the test group was normalized, and the remaining mRNA expression level of the target gene in the control group was defined as 100%.

$$Relative residual expression level of the target gene mR N A in the test group = {2}^{- Δ Δ Ct} ( test group ) \times 100 \%$$

$$Inhibition rate of the target gene mR N A in the test group = 100 \% - relative expression level of the target gene mR N A in the test group$$

The calculation method for the blood pressure detection data (blood pressure before the first administration (D0)) of the in-vivo activity experiments involved in the present disclosure is summarized as follows:

$$Blood pressure reduction level ( mmHg ) = Dx - D 0$$

The calculation method for the detection data of AGT and Ang II proteins at different time points in the in-vivo activity experiments involved in the present disclosure is summarized as follows:

$$Residual activity of the protein = \frac{{D}_{X}}{D 0} \times 100 \%$$

In the present disclosure, unless otherwise specified, all data from the in-vivo activity experiments are presented as X̅±SD(X±STDEV). All the experimental data are plotted and analyzed using GraphPad Prism 8.0 software.

### Example 1. In-Vitro Activity Evaluation of the Compound with (CR01008)×3 Conjugated to the 3′-End of the Sense Strand

The inhibitory activity of the CR01008 carrier-conjugated siRNA on the target gene AGT in cells was evaluated in the present Example using the target gene inhibitory activity assessment method in the human hepatocellular carcinoma cell line Huh7.

Preparation of test samples:
After centrifuging each of the above siRNA test samples, an appropriate amount of PBS was added according to the specification of each tube for dissolution to prepare a 20 μM stock solution. Then, the stock solution was further gradient-diluted with PBS to prepare 0.1 μM and 0.01 μM working solutions. Dose-response tests were conducted at final duplex concentrations of 1 nM and 0.1 nM.
Transfection and detection in 96-well plates:
Huh7 cells that had grown to near-confluence were digested with trypsin. The cells were washed to prepare a cell suspension. 100 μL of the cell suspension comprising 12,000 cells was added to each well of a 96-well plate and the plate was placed in an incubator at 37° C. with 5% CO₂ for culture. When the cells had adhered for 24 h, the DMEM medium in the 96-well plate was aspirated, and 80 μL of Opti-MEM™ medium was added to each well. Subsequently, the 96-well plate was placed back into the incubator for continued culture. 1 μL of the 0.1 μM and 0.01 μM working solutions was dispersed in 9 μL of Opti-MEM to form siRNA mixtures. 0.3 μL of RNAiMAX was dispersed in 9.7 μL of Opti-MEM and mixed with each siRNA mixture to form transfection complexes. The transfection complexes were incubated at room temperature for 10 minutes and then added to the 96-well plate at 20 μL/well. After 4 h of culture, 100 μL of DMEM medium comprising 20% FBS was supplemented to each well, and the 96-well plate was placed back into the incubator for another 24 h of culture. Wherein, in the Mock control group: 0.3 μL of RNAiMAX was dispersed in 9.7 μL of Opti-MEM, and then 10 μL of Opti-MEM was added. The mixture was incubated at room temperature for 10 minutes and then added to the 96-well plate at 20 μL/well. After 4 h of culture, 100 μL of DMEM medium comprising 20% FBS was supplemented to each well, and the 96-well plate was placed back into the incubator for another 24 h of culture. Then the 96-well plate was taken out, and the total RNA was obtained by being extracted according to the standard operating procedures for total RNA extraction using a fully automated nucleic acid extractor (purchased from Zhejiang Hanwei Technology Co., Ltd.) and a nucleic acid extraction kit (purchased from Zhejiang Hanwei Technology Co., Ltd., GO-MNTR-100).

The reverse transcription kit (Thermo Fisher scientific Company, RevertAid First Strand cDNA Synthesis Kit, K1622,) was used, and Oligo (dT)₁₈ reverse transcription primers were selected. A 20 μL reverse transcription system was prepared and the reverse transcription reaction was completed according to the method described in the instruction of the reverse transcription kit. Subsequently, the real-time fluorescent quantitative PCR kit (Thermo Fisher scientific Company, TaqMan Fast Advanced Master Mix, 4444557) was used to detect the expression level of the target gene mRNA in Hela cells on a fluorescent quantitative PCR instrument (LightCycler 480, from Roche company). In this real-time fluorescent quantitative PCR method, the glyceraldehyde-3-phosphate dehydrogenase (GAPDH) gene was used as the internal reference gene. Primers specific for the target gene and primers specific for the GAPDH internal reference gene were used to detect the target gene and the GAPDH internal reference gene respectively. The sequences of the detection primers are shown in Table 7.

TABLE 7
The sequences of the detection primers

| | | Primer | | Fluorescent |
|---|---|---|---|---|
| Gene | | type | Primer sequence | group |
| Target | AGT | Forward | 5′- | / |
| gene | | primer | GTATGTACACCCGGTCAC | |
| | | | C-3′ (SEQ ID No. 13) | |
| | | Reverse | 5′- | / |
| | | primer | GTTGTTCTGGGTACTACA | |
| | | | GCA-3′ (SEQ ID No. 14) | |
| | | Probe | 5′- | 5′FAM; |
| | | primer | CATCCTCTGCCTCCTGGCC | 3′MGB |
| | | | TG-3′ (SEQ ID No. 15) | |
| Internal | GAPDH | Forward | 5′- | / |
| reference | | primer | AAGAAGGTGGTGAAGCAG | |
| gene | | | G-3′ (SEQ ID No. 16) | |
| | | Reverse | 5′- | / |
| | | primer | CAAAGTGGTCGTTGAGGG-3′ | |
| | | | (SEQ ID No. 17) | |
| | | Probe | 5′- | 5′VIC; |
| | | primer | CAACAGCGACACCCACTC-3′ | 3′MGB |
| | | | (SEQ ID No. 18) | |

According to the method described in the instruction of the real-time fluorescent quantitative PCR kit, a 10 μL Real-time PCR reaction system was prepared for each PCR detection well. Each reaction system comprised 4 μL of the cDNA solution obtained from the above reverse transcription reaction, 5 μL of TaqMan™ Fast Advanced Master Mix (2×), 0.15 μL of 10 μM Forward primer, 0.15 μL of 10 μM Reverse primer, 0.15 μL of 10 μM Probe primer, and 0.55 μL of RNase-Free H₂O. The prepared reaction system was placed on a real-time fluorescent quantitative PCR instrument (Bio-Rad Company, CFX Opus 384), and Real-time PCR amplification was carried out using a two-step method. The amplification program was 2 min at 50° C., then pre-denaturation at 95° C. for 20s, denaturation at 95° C. for 3s, annealing and extension at 60° C. for 3s. The process of denaturation, annealing, and extension was repeated for 40 cycles. In the real-time fluorescent quantitative PCR method, the relative quantitative calculation of the expression level and inhibition rate of the target gene mRNA in each test group was carried out using the ΔΔCt method according to the technical method in the implementation mode.

TABLE 8
The inhibitory activity of the target gene in Huh7 cells
after administering the compounds of the present Example

| | |
|---|---|
| 1 nM | 0.1 nM |

| | % Residual | | % Residual | |
|---|---|---|---|---|
| Group | activity | STDEV | activity | STDEV |
| Mock | 100.00 | 4.01 | 100.00 | 4.01 |
| RZ003028 | 4.17 | 1.35 | 9.07 | 0.69 |
| RZ003029 | 3.98 | 0.27 | 15.74 | 0.34 |
| RZ003030 | 5.35 | 0.38 | 31.40 | 0.58 |
| RZ003031 | 3.19 | 0.21 | 17.31 | 0.18 |
| RZ003032 | 4.17 | 1.15 | 15.34 | 1.91 |
| RZ003041 | 4.77 | 0.27 | 20.13 | 1.25 |
| RZ003042 | 3.92 | 0.84 | 20.74 | 0.43 |
| RZ003043 | 5.23 | 0.44 | 34.10 | 5.74 |
| RZ003044 | 7.99 | 2.01 | 52.65 | 8.76 |
| RZ003045 | 7.79 | 0.86 | 43.83 | 0.32 |
| RZ003051 | 3.03 | 0.97 | 10.10 | 1.79 |
| RZ003052 | 3.50 | 0.68 | 11.81 | 0.91 |
| RZ003053 | 4.24 | 0.98 | 19.92 | 2.96 |
| RZ003054 | 3.85 | 0.61 | 18.55 | 1.00 |
| RZ003055 | 3.07 | 0.32 | 19.15 | 0.63 |

The results of Example 1 indicated that at doses of 1 nM and 0.1 nM, siRNAs of different modified CR01008 carrier conjugates could significantly inhibit the expression of AGT mRNA in Huh7 cells (Table 8).

### Example 2. Evaluation of the Antihypertensive Effect of the Compound with (CR01008)×3 Conjugated to the 3′-End of the Sense Strand in hREN×hAGT Hypertensive Mice

The present Example used a non-invasive blood pressure monitor for small animals to measure the systolic blood pressure of mice to evaluate the blood pressure-regulating effects of RZ003062, RZ003064, RZ003065, and RZ003066 in mice. Zilebesiran was used as a positive control.

Animal grouping, administration, and tissue sample collection:
hREN×hAGT Hypertensive mice aged 9-11 weeks (purchased from Cyagen Biosciences (Suzhou) Co., Ltd.) were grouped according to the systolic blood pressure levels (all male). Each test group was administered a predetermined dose of the drug conjugate, and a PBS control group was added. The drug dose for all mice was calculated based on the body weight. A single dose was administered via abdominal subcutaneous injection. Each drug conjugate was administered in the form of a 0.3 mg/mL (calculated as siRNA) PBS solution. The administration volume was 10 mL/kg of mouse body weight. That is, the administration dose of each drug conjugate was 3 mg/kg of mouse body weight (calculated as siRNA). The PBS control group was given the same volume of PBS solution (without the drug conjugate). The systolic blood pressure (SBP) was measured using a non-invasive blood pressure monitor for small animals (Ruanlong, BP-2010A) before administration (recorded as pre-dose, the value before administration), on the 3rd day after administration (recorded as D3), the 6th day (recorded as D6), the 10th day (recorded as D10), the 13th day (recorded as D13), the 17th day (recorded as D17), the 20th day (recorded as D20), the 24th day (recorded as D24), the 27th day (recorded as D27), and the 31st day (recorded as D31).

TABLE 9
Changes in systolic blood pressure levels in hREN × hAGT hypertensive
mice after administration of the compound according to the present Example

| | Pre-dose value | | | | | | | | | |
|---|---|---|---|---|---|---|---|---|---|---|
| | (mmHg) | | D 3(mmHg) | | D 6(mmHg) | | D 10(mmHg) | | D 13(mmHg) | |
| | Average | | Average | | Average | | Average | | Average | |
| Group | value | STDEV | value | STDEV | value | STDEV | value | STDEV | value | STDEV |
| PBS | 0.00 | 0.00 | 4.30 | 5.17 | 4.45 | 3.06 | 0.86 | 4.59 | 0.47 | 2.89 |
| Zilebesiran | 0.00 | 0.00 | −2.89 | 4.86 | −2.17 | 7.79 | −2.36 | 8.25 | −4.86 | 7.25 |
| RZ003062 | 0.00 | 0.00 | −1.66 | 12.41 | 2.23 | 13.21 | 1.65 | 12.01 | 1.03 | 10.53 |
| RZ003064 | 0.00 | 0.00 | −11.38 | 5.54 | −12.10 | 4.16 | −13.74 | 4.97 | −10.47 | 5.41 |
| RZ003065 | 0.00 | 0.00 | −9.10 | 6.65 | −10.61 | 5.73 | −8.82 | 7.12 | −10.31 | 7.33 |
| RZ003066 | 0.00 | 0.00 | −0.70 | 8.57 | −5.03 | 5.18 | −27.90 | 56.84 | −30.84 | 55.39 |
| | D 17(mmHg) | | D 20(mmHg) | | D 24(mmHg) | | D 27(mmHg) | | D 31(mmHg) | |
| | Average | | Average | | Average | | Average | | Average | |
| Group | value | STDEV | value | STDEV | value | STDEV | value | STDEV | value | STDEV |
| PBS | 1.44 | 5.86 | 0.48 | 5.78 | 0.94 | 7.55 | 1.08 | 6.61 | 1.60 | 6.11 |
| Zilebesiran | −1.39 | 8.75 | −1.29 | 7.58 | −3.04 | 9.10 | −0.48 | 8.39 | −1.25 | 7.31 |
| RZ003062 | 1.81 | 12.94 | 3.58 | 13.47 | 3.95 | 11.80 | 5.36 | 10.89 | 5.65 | 14.82 |
| RZ003064 | −9.82 | 5.28 | −7.61 | 6.28 | −7.25 | 4.85 | −5.89 | 4.00 | −3.49 | 3.29 |
| RZ003065 | −8.23 | 6.17 | −4.68 | 6.68 | −4.45 | 7.87 | −4.06 | 7.37 | −2.12 | 5.44 |
| RZ003066 | −8.70 | 5.36 | −8.04 | 5.10 | −7.94 | 5.53 | −2.77 | 2.71 | −1.03 | 5.30 |

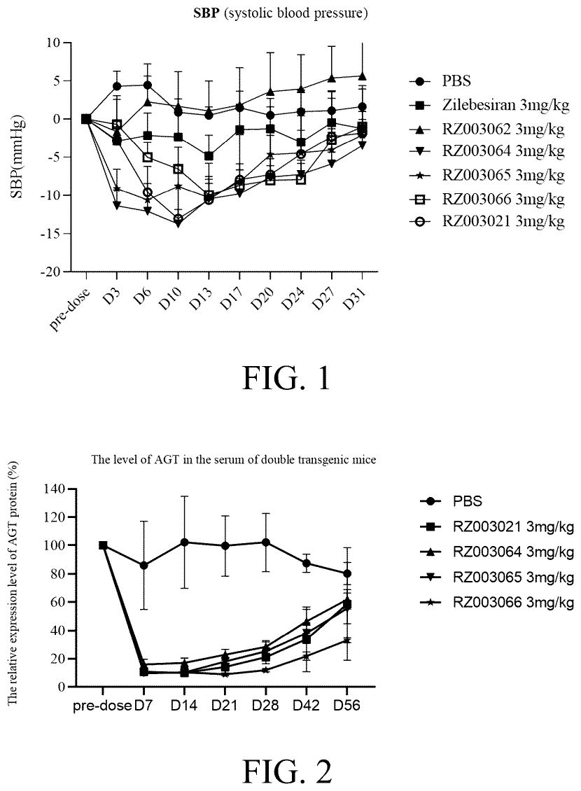

The results of Example 2 indicated that at a single-dose of 3 mg/kg, RZ003062, RZ003064, RZ003065, and RZ003066 could reduce the systolic blood pressure levels in hREN×hAGT hypertensive mice. As the observation time was extended, the blood pressure levels gradually recovered, while at the same time points, Zilebesiran had no obvious effect on reducing systolic blood pressure (Table 9, FIG. 1).

### Example 3. Evaluation of the Protein-Lowering Effect of the Compound with (CR01008)×3 Conjugated to the 3′-End of the Sense Strand in hREN×hAGT Hypertensive Mice

The present Example used the enzyme-linked immunosorbent assay (ELISA) to measure the expression of AGT protein in the serum of mice at different time points after a single administration of the CR01008 carrier conjugates RZ003064, RZ003065, and RZ003066.

Animal grouping, administration, and tissue sample collection: hREN×hAGT Hypertensive mice aged 9-11 weeks (purchased from Cyagen Biosciences (Suzhou) Co., Ltd.) were grouped according to the systolic blood pressure levels (all male). Each test group was administered a predetermined dose of the drug conjugate, and a PBS control group was added. The drug dose for all mice was calculated based on the body weight. A single dose was administered via abdominal subcutaneous injection. Each drug conjugate was administered in the form of a 0.3 mg/mL (calculated as siRNA) PBS solution. The administration volume was 10 mL/kg of mouse body weight. That is, the administration dose of each drug conjugate was 3 mg/kg of mouse body weight (calculated as siRNA). The PBS control group was given the same volume of PBS solution (without the drug conjugate). Serum samples were collected from all groups of mice before administration (recorded as pre-dose, the value before administration), on the 7th day after administration (recorded as D7), the 14th day (recorded as D14), the 21st day (recorded as D21), the 28th day (recorded as D28), the 42nd day (recorded as D42), and the 56th day (recorded as D56). The expression of AGT protein was measured using a human angiotensinogen kit (IBL, 27412).

TABLE 10
Changes in human AGT protein levels in hREN × hAGT hypertensive
mice after administration of the compound according to the present Example

| | Pre-dose | | | | | | |
|---|---|---|---|---|---|---|---|
| | value | | Day 7 | | Day 14 | | Day 21 |
| | % | | % | | % | | % |
| | Residual | | Residual | | Residual | | Residual |
| Group | activity | STDEV | activity | STDEV | activity | STDEV | activity |
| PBS | 100.00 | 0.00 | 85.92 | 31.19 | 102.09 | 32.50 | 99.64 |
| RZ003064 | 100.00 | 0.00 | 15.89 | 3.76 | 17.06 | 3.36 | 22.77 |
| RZ003065 | 100.00 | 0.00 | 9.94 | 1.79 | 10.35 | 1.46 | 18.01 |
| RZ003066 | 100.00 | 0.00 | 9.94 | 0.68 | 10.46 | 1.06 | 9.01 |

| | | |
|---|---|---|
| Day 28 | Day 42 | Day 56 |

| | | % | | % | | % | |
|---|---|---|---|---|---|---|---|
| | Day 21 | Residual | | Residual | | Residual | |
| Group | STDEV | activity | STDEV | activity | STDEV | activity | STDEV |
| PBS | 21.27 | 102.10 | 20.61 | 87.40 | 6.38 | 80.16 | 7.82 |
| RZ003064 | 3.84 | 28.26 | 4.41 | 46.43 | 8.49 | 61.82 | 6.83 |
| RZ003065 | 2.87 | 25.15 | 6.48 | 38.05 | 7.26 | 55.51 | 10.75 |
| RZ003066 | 0.97 | 11.96 | 1.11 | 21.75 | 2.95 | 32.93 | 1.76 |

The results of Example 3 indicated that at a single-dose of 3 mg/kg, RZ003064, RZ003065, RZ003066, and RZ003021 all could significantly reduce the AGT protein level in hREN×hAGT hypertensive mice. By the 28th day of observation, the reduction in AGT protein level was still more than 70% (FIG. 2, Table 10).

The above specific embodiments are merely illustrative of the content of the present disclosure and do not represent a limitation of the content of the present disclosure. For those ordinary skilled in the art, various modifications and improvements can be made without departing from the spirit and essence of the present disclosure, and these modifications and improvements are also considered to fall within the protection scope of the present disclosure.

1. A compound having a structure represented by Formula (I) or a pharmaceutically acceptable salt thereof: 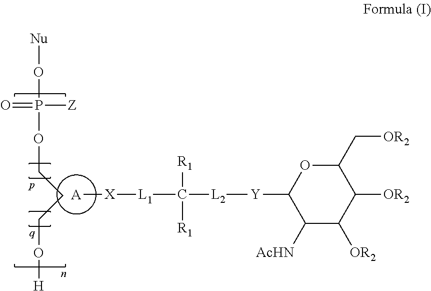
wherein, in the structure of the Formula mentioned above:
each A is independently selected from unsubstituted or substituted 4-10-membered aliphatic rings;
n is selected from 1, 2, 3 or 4;
each Z is independently hydroxyl or sulfydryl;
each p is independently selected from 1, 2 or 3;
each q is independently selected from 1, 2 or 3;
each X is independently selected from NH, O or S;
each L₁ is independently selected from 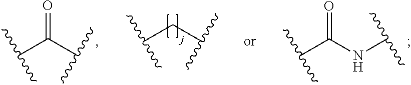
wherein, j is selected from 1, 2, 3, 4, 5, 6, 7, 8, 9 or 10;
each R₁ is independently selected from H, C₁-C₆ alkyl, C₁-C₆ halogenated alkyl or C₁-C₆ alkoxy;
each L₂ is independently selected from C₁-C₃₀ alkylene or 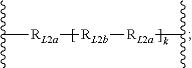
wherein, each R_{L2a} is independently selected from C₁-C₁₀ alkylene, each R_{L2b} is independently selected from O, S, NH or —NH—C(O)—, k is selected from 1, 2, 3, 4, 5, 6, 7, 8, 9 or 10;
each Y is independently selected from NH, O or S;
each R₂ is independently selected from H, 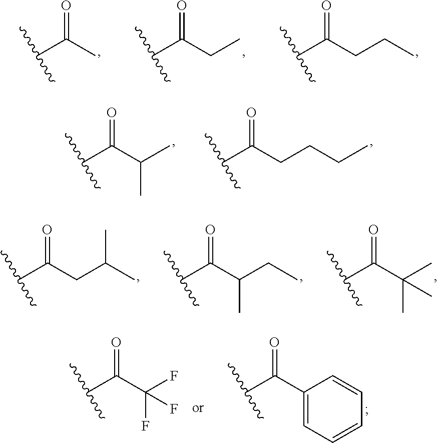
wherein, Nu represents a double-stranded oligonucleotide or a pharmaceutically acceptable salts thereof used for inhibiting the expression of AGT gene in a cell; the double-stranded oligonucleotide comprises a sense strand and an antisense strand, the sense strand and the antisense strand form a duplex region, and the antisense strand comprises a complementary sequence that is complementary to the AGT mRNA target sequence, the target sequence is selected from a nucleotide region consisting of 14-35 consecutive nucleotides on AGT mRNA.

2. The compound according to claim 1, wherein the compound has a structure represented by Formula (II) or a pharmaceutically acceptable salt thereof: 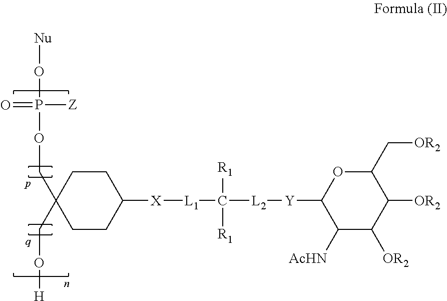
in Formula (II), p, q, n, Z, X, Y, L₁, L₂ and the substituent R₁ are as defined in claim 1, R₂ is selected from H.

3. The compound according to claim 1, wherein the compound has a structure represented by Formula (III) or a pharmaceutically acceptable salt thereof: 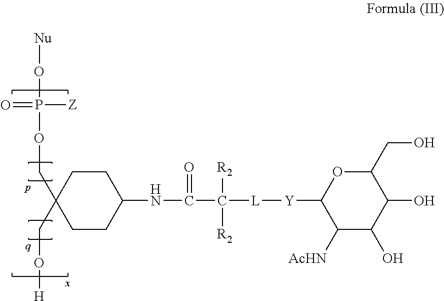
in Formula (III), m is selected from 1, 2, 3 or 4; the remaining substituents are as defined in claim 1;
further, the compound has a structure represented by Formula (IV) or a pharmaceutically acceptable salt thereof: 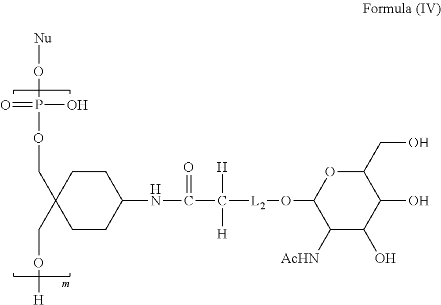
in Formula (IV), Nu is as defined in claim 1, m is selected from 1, 2, 3 or 4; L₂ is independently selected from

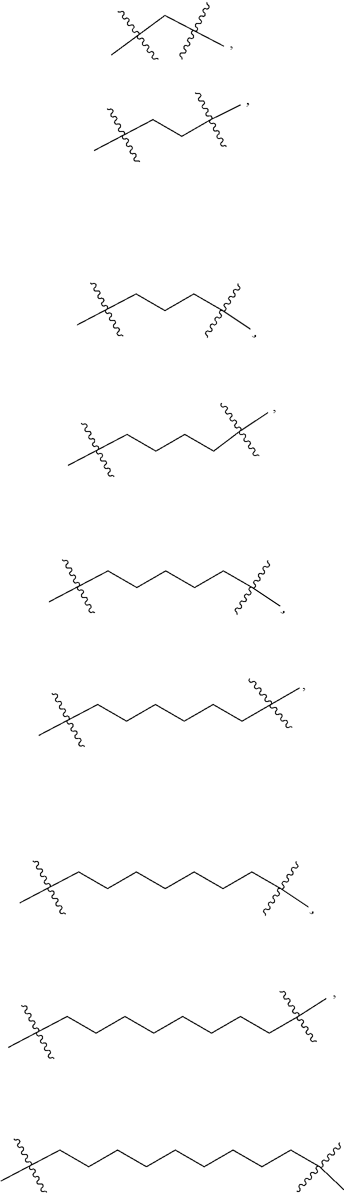

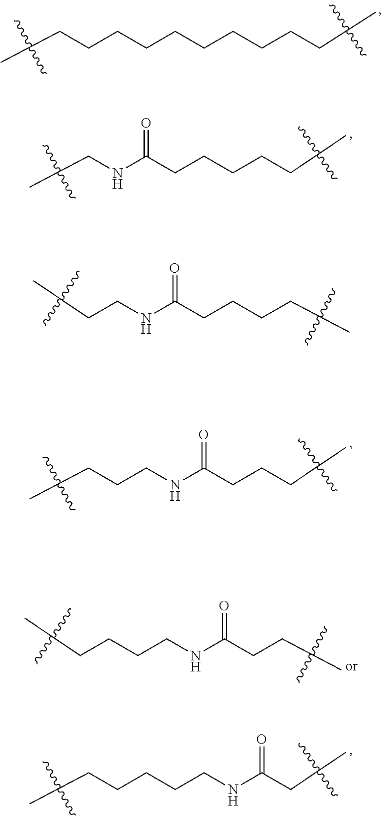 [SMILES: *CCCCCCCCCC* ; *CCCCCC(=O)NC* ; *CCCCC(=O)NCC* ; *CCCNC(=O)CCC* ; *CCCCNC(=O)CC* ; *CCCCCNC(=O)C*]

4. The compound according to claim 1,
wherein the compound has a structure represented by the following Formula or a pharmaceutically acceptable salt thereof: 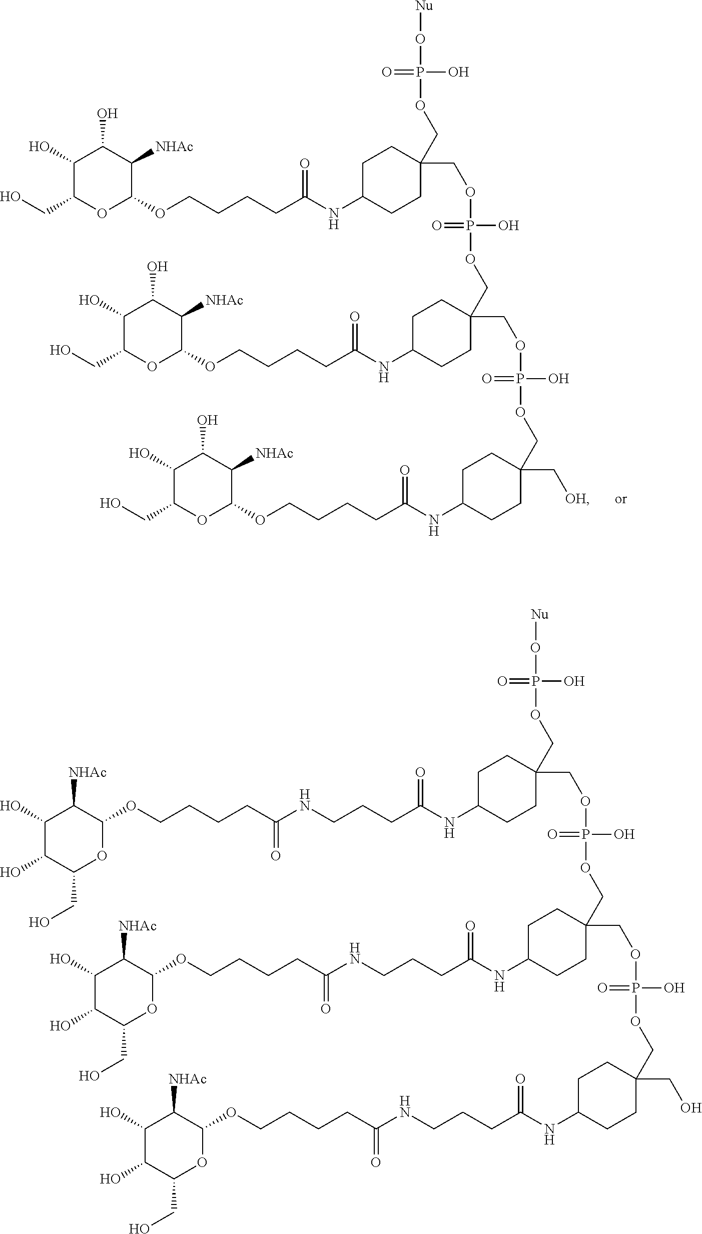
Nu is as defined in claim 1, and in the above oligonucleotide-conjugated compound, the 3′-end of the sense strand of Nu is connected to a phosphate group.

5. The compound according to claim 1, wherein,
in the double-stranded oligonucleotide represented by Nu, the sense strand comprises a nucleotide sequence of at least 17, at least 18, or at least 19 consecutive nucleotides in any one of the sequences of SEQ ID NO: 1, 3, 5, 7, 9, or 11, or comprises a nucleotide sequence having 1, 2, or 3 nucleotide differences from the consecutive nucleotides; and/or the antisense strand comprises a nucleotide sequence of at least 17, at least 18, or at least 19 consecutive nucleotides in any one of the sequences of SEQ ID NO: 2, 4, 6, 8, 10, or 12, or comprises a nucleotide sequence having 1, 2, or 3 nucleotide differences from the consecutive nucleotides;
optionally, in the double-stranded oligonucleotide represented by Nu, in the direction of 5′-3′, the antisense strand comprises the 1st-19th consecutive nucleotides in any one of the sequences of SEQ ID NO:2, 4, 6, 8, 10, or 12, or comprises a nucleotide sequence having 1, 2, or 3 nucleotide differences from the consecutive nucleotides;
optionally, in the double-stranded oligonucleotide represented by Nu, the sense strand comprises any one of the sequences of SEQ ID NO. 1, 3, 5, 7, 9, or 11, or a nucleotide sequence having 1 or 2 nucleotide differences from any of the above sequences; and/or, the antisense strand of the double-stranded oligonucleotide comprises any one of the sequences shown in SEQ ID NO. 2, 4, 6, 8, 10, or 12, or a nucleotide sequence having 1 or 2 nucleotide differences from any of the above sequences;
optionally, the double-stranded oligonucleotide is selected from one or more of the following groups:
1) the antisense strand has the nucleotide sequence shown in SEQ ID NO. 2 or a nucleotide sequence having 1 or 2 nucleotide differences therefrom; the sense strand has the nucleotide sequence shown in SEQ ID NO. 1 or a nucleotide sequence having 1 or 2 nucleotide differences therefrom;
2) the antisense strand has the nucleotide sequence shown in SEQ ID NO. 4 or a nucleotide sequence having 1 or 2 nucleotide differences therefrom; the sense strand has the nucleotide sequence shown in SEQ ID NO. 3 or a nucleotide sequence having 1 or 2 nucleotide differences therefrom;
3) the antisense strand has the nucleotide sequence shown in SEQ ID NO. 6 or a nucleotide sequence having 1 or 2 nucleotide differences therefrom; the sense strand has the nucleotide sequence shown in SEQ ID NO. 5 or a nucleotide sequence having 1 or 2 nucleotide differences therefrom;
4) the antisense strand has the nucleotide sequence shown in SEQ ID NO. 8 or a nucleotide sequence having 1 or 2 nucleotide differences therefrom; the sense strand has the nucleotide sequence shown in SEQ ID NO. 7 or a nucleotide sequence having 1 or 2 nucleotide differences therefrom;
5) the antisense strand has the nucleotide sequence shown in SEQ ID NO. 10 or a nucleotide sequence having 1 or 2 nucleotide differences therefrom; the sense strand has the nucleotide sequence shown in SEQ ID NO. 9 or a nucleotide sequence having 1 or 2 nucleotide differences therefrom;
6) the antisense strand has the nucleotide sequence shown in SEQ ID NO. 12 or a nucleotide sequence having 1 or 2 nucleotide differences therefrom; the sense strand has the nucleotide sequence shown in SEQ ID NO. 11 or a nucleotide sequence having 1 or 2 nucleotide differences therefrom.

6. The compound according to claim 1, wherein each nucleotide in the double-stranded oligonucleotide is independently selected from the following modified nucleotides:
2′-fluoro-modified nucleotides, 2′-deoxy-modified nucleotides, 2′-O-methyl-modified nucleotides, 2′-O—(CH₂)ₓ—O—Rₘ-modified nucleotides, 2′-O—Si(Rₙ)₃-modified nucleotides, 2′-amino-modified nucleotides, abasic nucleotides or nucleotide analogs; the nucleotide analogs are selected from one or more of PNA, MNA, BNA, LNA, GNA, TNA or UNA; wherein, x is selected from 1 or 2, Rₘ is selected from optionally substituted C₁₋₆ alkyl or optionally substituted C₁₋₆ alkoxy; if Rₘ comprises a substituent, the substituent is selected from halogen, C₁₋₆ alkoxy, hydroxyl or amino; Rₙ is independently selected from optionally substituted C₁₋₆ alkyl;
optionally, the 2′-O—(CH₂)ₓ—O—Rₘ-modified nucleotides are selected from 2′-O-methoxyethyl-modified nucleotides or 2′-O-ethoxyethyl-modified nucleotides;
optionally, the 2′-O—Si(Rₙ)₃ modified nucleotides are selected from 2′-O-TBDMS-modified nucleotides, 2′-O-TIPS-modified nucleotides or 2′-O-TOM-modified nucleotides;
optionally, the double-stranded oligonucleotide comprises at least one 2′-O-methoxyethyl-modified nucleotide.

7. The compound according to claim 1, wherein,
in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from nucleotides other than 2′-fluoro-modified nucleotides; at least four nucleotides among the nucleotides at positions 2nd, 6th, 9th-12th, 14th and 16th of the nucleotide sequence in the antisense strand are selected from 2′-fluoro-modified nucleotides, and the nucleotides at the remaining positions are independently selected from nucleotides other than 2′-fluoro-modified nucleotides;
optionally, in the direction from the 5′-end to the 3′-end, at least three nucleotides among the nucleotides at positions 7th-10th of the nucleotide sequence in the sense strand are selected from 2′-fluoro-modified nucleotides, and the nucleotides at the remaining positions are independently selected from 2′-O-methyl-modified nucleotides or 2′-O-methoxyethyl-modified nucleotides; at least four nucleotides among the nucleotides at positions 2nd, 6th, 9th-12th, 14th and 16th of the nucleotide sequence in the antisense strand are selected from 2′-fluoro-modified nucleotides, and the nucleotides at the remaining positions are selected from 2′-O-methyl-modified nucleotides or 2′-O-methoxyethyl-modified nucleotides;
optionally, in the direction from the 5′-end to the 3′-end, at least one of the linkages between the following nucleotides of the sense strand is a phosphorothioate linkage: the linkage between the first and the second nucleotides at the 5′-end of the sense strand; the linkage between the second and the third nucleotides at the 5′-end of the sense strand;
optionally, in the direction from the 5′-end to the 3′-end, at least one of the linkages between the following nucleotides of the antisense strand is a phosphorothioate linkage: the linkage between the first and the second nucleotides at the 5′-end of the antisense strand; the linkage between the second and the third nucleotides at the 5′-end of the antisense strand; the linkage between the first and the second nucleotides at the 3′-end of the antisense strand; the linkage between the second and the third nucleotides at the 3′-end of the antisense strand.

8. The compound according to claim 1, wherein each nucleotide in the double-stranded oligonucleotide is selected from modified nucleotides;
optionally, the double-stranded oligonucleotide is selected from at least one of the following groups:

| | sense strand (5′-3′) | antisense strand (5′-3′) |
|---|---|---|
| group 1 | CmsAmsAmGmUmUmGfAfGfAf | AmsAfsUmUmUmUfUmGmUmUfCm |
| | AmCmAmAmAmAmAmUmUm | UmCmAfA(moe)CfUmUmGmsAmsAm |
| group 2 | GmsUmsUmUmUmAmAfAfAfUf | UmsAfsUmAmCmUfUmUmAmAmUf |
| | UmAmAmAmGmUmAmUmAm | UmUmUfA(moe)AfAmAmCmsCmsCm |
| group 3 | AmsAmsGmUmAmUmAfCfAfUf | AmsAfsUmGmCmAfAmAmAmAfUm |
| | UmUmUmUmGmCmAmUmUm | GmUmAfUmAfCmUmUmsUmsAm |
| group 4 | AmsAmsGmUmAmUmAfCfAfUf | AmsAfsUmGmCmAfAmAmAmAfUm |
| | UmUmUmUmGmCmAmUmUm | GmUmAfT(moe)AfCmUmUmsUmsAm. |

9. The compound according to claim 1, wherein the compound is selected from any of RZ003028, RZ003029, RZ003030, RZ003031, RZ003032, RZ003041, RZ003042, RZ003043, RZ003044, RZ003045, RZ003051, RZ003052, RZ003053, RZ003054, RZ003055, RZ003062, RZ003064, RZ003065, RZ003066, RZ003069, RZ003070, RZ003071, RZ003072, RZ003073, RZ003074, RZ003075, RZ003076, RZ003077, RZ003078, RZ003079, RZ003080, RZ003081, RZ003082, RZ003083, RZ003084, RZ003085, RZ003086, RZ003087, RZ003088, RZ003089, RZ003090, RZ003091, RZ003092, RZ003093, or RZ003094 as shown in the following table:

| | sense strand (5′-3′) | antisense strand (5′-3′) |
|---|---|---|
| RZ003028 | CmsAmsAmGmUmUmGfAfGfAfAmC(moe) | AmsAfsUmUmUmUfUmGmUmUf |
| | AmAmAmAmAmUmUm_{—} | CmUmCmAfA(moe)CfUmUmGms |
| | (CR01008 × 3) | AmsAm |
| RZ003029 | CmsAmsAmGmUmUmGfAfGfAfAmC(moe) | AmsAfsUmUmUmUfUmGmUmU |
| | AmAmAmAmAmUmUm_{—} | mCfUmCmAfA(moe)CfUmUmGm |
| | (CR01008 × 3) | sAmsAm |
| RZ003030 | CmsAmsAmGmUmUmGfAfGfAfAmC | AmsAfsUmUmUmUfUmGmUmUf |
| | mAmAmAmAmAmT(moe)Um_{—} | CmUmCmAfA(moe)CfUmUmGms |
| | (CR01008 × 3) | AmsAm |
| RZ003031 | CmsAmsAmGmUmUmGfAfGfAfAmC(moe) | AmsAfsUmUmUmUfUmGmUmUf |
| | AmAmAmAmAmT(moe)Um_{—} | CmUmCmAfA(moe)CfUmUmGms |
| | (CR01008 × 3) | AmsAm |
| RZ003032 | CmsAmsAmGmUmUmGfAfGfAfAmC | AmsAfsUmUmUmUfUmG(moe)U |
| | mAmAmAmAmAmT(moe)Um_{—} | mUfCmUmCmAfAmCfUmUmGms |
| | (CR01008 × 3) | AmsAm |
| RZ003041 | GmsUmsUmUmUmAmAfAfAfUfUmA | UmsAfsUmAmCmUfUmUmAmAf |
| | (moe)AmAmGmUmAmUmAm_{—} | UmUmUmUfA(moe)AfAmAmCms |
| | (CR01008 × 3) | CmsCm |
| RZ003042 | GmsUmsUmUmUmAmAfAfAfUfUmA | UmsAfsUmAmCmUfUmUmAmA |
| | (moe)AmAmGmUmAmUmAm_{—} | mUfUmUmUfA(moe)AfAmAmCm |
| | (CR01008 × 3) | sCmsCm |
| RZ003043 | GmsUmsUmUmUmAmAfAfAfUfUmA | UmsAfsUmAmCmUfUmUmAmAf |
| | mAmAmGmUmAmT(moe)Am_{—} | UmUmUmUfA(moe)AfAmAmCms |
| | (CR01008 × 3) | CmsCm |
| RZ003044 | GmsUmsUmUmUmAmAfAfAfUfUmA | UmsAfsUmAmCmUfUmUmAmAf |
| | (moe)AmAmGmUmAmT(moe)Am_{—} | UmUmUmUfA(moe)AfAmAmCms |
| | (CR01008 × 3) | CmsCm |
| RZ003045 | GmsUmsUmUmUmAmAfAfAfUfUmA | UmsAfsUmAmCmUfUmT(moe)A |
| | mAmAmGmUmAmT(moe)Am_{—} | mAfUmUmUmUfAmAfAmAmCm |
| | (CR01008 × 3) | sCmsCm |
| RZ003051 | AmsAmsGmUmAmUmAfCfAfUfUmT(moe) | AmsAfsUmGmCmAfAmAmAmA |
| | UmUmGmCmAmUmUm_{—} | mUmGmUmAfT(moe)AfCmUmU |
| | (CR01008 × 3) | msUmsAm |
| RZ003052 | AmsAmsGmUmAmUmAfCfAfUfUmT(moe) | AmsAfsUmGmCmAfAmAmAmAf |
| | UmUmGmCmAmUmUm_{—} | UmGmUmAfT(moe)AfCmUmUms |
| | (CR01008 × 3) | UmsAm |
| RZ003053 | AmsAmsGmUmAmUmAfCfAfUfUmU | AmsAfsUmGmCmAfAmAmAmA |
| | mUmUmGmCmAmT(moe)Um_{—} | mUfGmUmAfT(moe)AfCmUmUm |
| | (CR01008 × 3) | sUmsAm |
| RZ003054 | AmsAmsGmUmAmUmAfCfAfUfUmT(moe) | AmsAfsUmGmCmAfAmAmAmAf |
| | UmUmGmCmAmT(moe)Um_{—} | UmGmUmAfT(moe)AfCmUmUms |
| | (CR01008 × 3) | UmsAm |
| RZ003055 | AmsAmsGmUmAmUmAfCfAfUfUmU | AmsAfsUmGmCmAfAmAmAmAf |
| | mUmUmGmCmAmT(moe)Um_{—} | UmGmUmAfT(moe)AfCmUmUms |
| | (CR01008 × 3) | UmsAm |
| RZ003062 | CmsAmsAmGmUmUmGfAfGfAfAmC | AmsAfsUmUmUmUfUmGmUmUf |
| | mAmAmAmAmAmUmUm_(CR01008 × | CmUmCmAfA(moe)CfUmUmGms |
| | 3) | AmsAm |
| RZ003064 | GmsUmsUmUmUmAmAfAfAfUfUmA | UmsAfsUmAmCmUfUmUmAmA |
| | mAmAmGmUmAmUmAm_(CR01008 × | mUfUmUmUfA(moe)AfAmAmCm |
| | 3) | sCmsCm |
| RZ003065 | AmsAmsGmUmAmUmAfCfAfUfUmU | AmsAfsUmGmCmAfAmAmAmAf |
| | mUmUmGmCmAmUmUm_(CR01008 × | UmGmUmAfUmAfCmUmUmsUm |
| | 3) | sAm |
| RZ003066 | AmsAmsGmUmAmUmAfCfAfUfUmU | AmsAfsUmGmCmAfAmAmAmAf |
| | mUmUmGmCmAmUmUm_(CR01008 × | UmGmUmAfT(moe)AfCmUmUms |
| | 3) | UmsAm |
| RZ003069 | GmsUmsUmUmUmAmAfAfAfUfUmA | UmsAfsUmAmCmUfUmUmAfAm |
| | mAmAmGmUmAmUmAm_(CR01008 × | UmUmUmUfA(moe)AfAmAmCms |
| | 3) | CmsCm |
| RZ003070 | GmsUmsUmUmUmAmAfAfAfUfUmA | UmsAfsUmAmCmUfUmUmAmA |
| | mAmAmGmUmAmUmAm_(CR01008 × | mUmUfUmUfA(moe)AfAmAmCm |
| | 3) | sCmsCm |
| RZ003071 | AmsAmsGmUmAmUmAfCfAfUfUmU | AmsAfsUmGmCmAfAmAmAfAm |
| | mUmUmGmCmAmUmUm_(CR01008 × | UmGmUmAfT(moe)AfCmUmUms |
| | 3) | UmsAm |
| RZ003072 | AmsAmsGmUmAmUmAfCfAfUfUmU | AmsAfsUmGmCmAfAmAmAmA |
| | mUmUmGmCmAmUmUm_(CR01008 × | mUmGfUmAfT(moe)AfCmUmUm |
| | 3) | sUmsAm |
| RZ003073 | AmsAmsGmUmAmUmAfCfAfUfUmU | AmsAfsUmGmCmAfAmAmAfAm |
| | mUmUmGmCmAmUmUm_(CR01008 × | UmGmUmAfU(moe)AfCmUmUms |
| | 3) | UmsAm |
| RZ003074 | AmsAmsGmUmAmUmAfCfAfUfUmU | AmsAfsUmGmCmAfAmAmAmA |
| | mUmUmGmCmAmUmUm_(CR01008 × | mUmGfUmAfU(moe)AfCmUmUm |
| | 3) | sUmsAm |
| RZ003075 | CmsAmsAmGmUmUmGfAfGfAfAmC | AmsAfsUmUmUmUfUmGmUmU |
| | mAmAmAmAmAmUmUm_(CR01008 × | mCmUfCmAfA(moe)CfUmUmGm |
| | 3) | sAmsAm |
| RZ003076 | CmsAmsAmGmUmUmGfAfGfAfAmC | AmsAfsUmUmUmUfUmGmUfUm |
| | mAmAmAmAmAmUmUm_(CR01008 × | CmUmCmAfA(moe)CfUmUmGms |
| | 3 | AmsAm |
| RZ003077 | CmsAmsAmGmUmUmGfAfGfAfAmC | AmsAfsUmUmUmUfUmGmUmU |
| | mAmAmAmAmAmUmUm_(CR01008 × | mCmUfCmAfAmCfUmUmGmsAm |
| | 3 | sAm |
| RZ003078 | CmsAmsAmGmUmUmGfAfGfAfAmC | AmsAfsUmUmUmUfUmGmUfUm |
| | mAmAmAmAmAmUmUm_(CR01008 × | CmUmCmAfAmCfUmUmGmsAm |
| | 3 | sAm |
| RZ003079 | GmsUmsUmUmUmAmAfAfAfUfUmA | UmsAfsUmAmCmUfUmUmAfAm |
| | mAmAmGmUmAmUmAm_(CR01008 × | UmUmUmUfAmAfAmAmCmsCm |
| | 3 | sCm |
| RZ003080 | GmsUmsUmUmUmAmAfAfAfUfUmA | UmsAfsUmAmCmUfUmUmAmA |
| | mAmAmGmUmAmUmAm_(CR01008 × | mUmUfUmUfAmAfAmAmCmsC |
| | 3 | msCm |
| RZ003081 | AmsAmsGmUmAmUmAfCfAfUfUmU | AmsAfsUmGmCmAfAmAmAfAm |
| | mUmUmGmCmAmUmUm_(CR01008 × | UmGmUmAfUmAfCmUmUmsUm |
| | 3) | sAm |
| RZ003082 | AmsAmsGmUmAmUmAfCfAfUfUmU | AmsAfsUmGmCmAfAmAmAmA |
| | mUmUmGmCmAmUmUm_(CR01008 × | mUmGfUmAfUmAfCmUmUmsU |
| | 3) | msAm |
| RZ003083 | CmsAmsUmCmCmAmCfAfAfUfGmA | GmsGfsUmAmCmUfCmUmCfAm |
| | mGmAmGmUmAmCmCm_(CR01008 × | UmUmGmUfGmGfAmUmGmsAm |
| | 3) | sCm |
| RZ003084 | CmsAmsUmCmCmAmCfAfAfUfGmA | GmsGfsUmAmCmUfCmUmCmA |
| | mGmAmGmUmAmCmCm_(CR01008 × | mUmUfGmUfGmGfAmUmGmsA |
| | 3) | msCm |
| RZ003085 | CmsAmsUmCmCmAmCfAfAfUfGmA | GmsGfsUmAmCmUfCmUmCfAm |
| | mGmAmGmUmAmCmCm_(CR01008 × | UmUmGmUfG(moe)GfAmUmGms |
| | 3) | AmsCm |
| RZ003086 | CmsAmsUmCmCmAmCfAfAfUfGmA | GmsGfsUmAmCmUfCmUmCmA |
| | mGmAmGmUmAmCmCm_(CR01008 × | mUmUfGmUfG(moe)GfAmUmGm |
| | 3) | sAmsCm |
| RZ003087 | UmsCmsAmAmCmUmGfGfAfUfGmA | AmsGfsUmUmUmCfUmUmCfAm |
| | mAmGmAmAmAmCmUm_(CR01008 × | UmCmCmAfGmUfUmGmAmsGm |
| | 3) | sGm |
| RZ003088 | UmsCmsAmAmCmUmGfGfAfUfGmA | AmsGfsUmUmUmCfUmUmCmA |
| | mAmGmAmAmAmCmUm_(CR01008 × | mUmCfCmAfGmUfUmGmAmsG |
| | 3) | msGm |
| RZ003089 | UmsCmsAmAmCmUmGfGfAfUfGmA | AmsGfsUmUmUmCfUmUmCfAm |
| | mAmGmAmAmAmCmUm_(CR01008 × | UmCmCmAfG(moe)UfUmGmAms |
| | 3) | GmsGm |
| RZ003090 | UmsCmsAmAmCmUmGfGfAfUfGmA | AmsGfsUmUmUmCfUmUmCmA |
| | mAmGmAmAmAmCmUm_(CR01008 × | mUmCfCmAfG(moe)UfUmGmAm |
| | 3) | sGmsGm |
| RZ003091 | CmsCmsUmGmUmUmUfGfCfUfGmU | AmsUfsCmAmUmAfCmAmCfAm |
| | mGmUmAmUmGmAmUm_(CR01008 × | GmCmAmAfAmCfAmGmGmsAm |
| | 3) | sAm |
| RZ003092 | CmsCmsUmGmUmUmUfGfCfUfGmU | AmsUfsCmAmUmAfCmAmCmA |
| | mGmUmAmUmGmAmUm_(CR01008 × | mGmCfAmAfAmCfAmGmGmsA |
| | 3) | msAm |
| RZ003093 | CmsCmsUmGmUmUmUfGfCfUfGmU | AmsUfsCmAmUmAfCmAmCfAm |
| | mGmUmAmUmGmAmUm_(CR01008 × | GmCmAmAfA(moe)CfAmGmGms |
| | 3) | AmsAm |
| RZ003094 | CmsCmsUmGmUmUmUfGfCfUfGmU | AmsUfsCmAmUmAfCmAmCmA |
| | mGmUmAmUmGmAmUm_(CR01008 × | mGmCfAmAfA(moe)CfAmGmGm |
| | 3) | sAmsAm; |

optionally, the compound is selected from any one of RZ003062, RZ003064, RZ003065,_RZ003066,_or RZ003089:

| | sense strand (5′-3′) | antisense strand (5′-3′) |
|---|---|---|
| RZ003062 | CmsAmsAmGmUmUmGfAfGfAf | AmsAfsUmUmUmUfUmGmUmUfC |
| | AmCmAmAmAmAmAmUmUm_{—} | mUmCmAfA(moe)CfUmUmGmsA |
| | (CR01008 × 3) | msAm |
| RZ003064 | GmsUmsUmUmUmAmAfAfAfUf | UmsAfsUmAmCmUfUmUmAmAm |
| | UmAmAmAmGmUmAmUmAm_{—} | UfUmUmUfA(moe)AfAmAmCmsC |
| | (CR01008 × 3) | msCm |
| RZ003065 | AmsAmsGmUmAmUmAfCfAfUf | AmsAfsUmGmCmAfAmAmAmAfU |
| | UmUmUmUmGmCmAmUmUm_{—} | mGmUmAfUmAfCmUmUmsUmsA |
| | (CR01008 × 3) | m |
| RZ003066 | AmsAmsGmUmAmUmAfCfAfUf | AmsAfsUmGmCmAfAmAmAmAfU |
| | UmUmUmUmGmCmAmUmUm_{—} | mGmUmAfT(moe)AfCmUmUmsUm |
| | (CR01008 × 3) | sAm |
| RZ003089 | UmsCmsAmAmCmUmGfGfAfUf | AmsGfsUmUmUmCfUmUmCfAmU |
| | GmAmAmGmAmAmAmCmUm_{—} | mCmCmAfG(moe)UfUmGmAmsG |
| | (CR01008 × 3) | msGm. |

10. A pharmaceutical composition, wherein the pharmaceutical composition comprises the compound according to claim 1 and a pharmaceutically acceptable excipient.

11. (canceled)

12. A kit, wherein the kit comprises the compound according to claim 1 or a composition comprising the compound according to claim 1 and a pharmaceutically acceptable excipient.

13. A method for inhibiting the expression of AGT in a cell, wherein the method comprises administering to a subject the compound according to claim 1 or administering a composition comprising the compound according to claim 1 and a pharmaceutically acceptable excipient.

14. A method for alleviating, preventing and/or treating a disease or disorder mediated by AGT, wherein the method comprises administering to a subject the compound according to claim 1 or administering a composition comprising the compound according to claim 1 and a pharmaceutically acceptable excipient;
optionally, the disease or disorder mediated by AGT includes a disease associated with the mRNA level of the AGT gene expression;
optionally, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

15. A pharmaceutical composition, wherein the pharmaceutical composition comprises the compound according to claim 2 and a pharmaceutically acceptable excipient.

16. A pharmaceutical composition, wherein the pharmaceutical composition comprises the compound according to claim 3 and a pharmaceutically acceptable excipient.

17. A pharmaceutical composition, wherein the pharmaceutical composition comprises the compound according to claim 4 and a pharmaceutically acceptable excipient.

18. A method for inhibiting the expression of AGT in a cell, wherein the method comprises administering to a subject the compound according to claim 4 or administering a composition comprising the compound according to claim 4 and a pharmaceutically acceptable excipient.

19. A method for alleviating, preventing and/or treating a disease or disorder mediated by AGT, wherein the method comprises administering to a subject the compound according to claim 2 or administering a composition comprising the compound according to claim 2 and a pharmaceutically acceptable excipient;
optionally, the disease or disorder mediated by AGT includes a disease associated with the mRNA level of the AGT gene expression;
optionally, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

20. A method for alleviating, preventing and/or treating a disease or disorder mediated by AGT, wherein the method comprises administering to a subject the compound according to claim 3 or administering a composition comprising the compound according to claim 3 and a pharmaceutically acceptable excipient;
optionally, the disease or disorder mediated by AGT includes a disease associated with the mRNA level of the AGT gene expression;
optionally, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.

21. A method for alleviating, preventing and/or treating a disease or disorder mediated by AGT, wherein the method comprises administering to a subject the compound according to claim 4 or administering a composition comprising the compound according to claim 4 and a pharmaceutically acceptable excipient;
optionally, the disease or disorder mediated by AGT includes a disease associated with the mRNA level of the AGT gene expression;
optionally, the disease or disorder includes hypertension, borderline hypertension, essential hypertension, secondary hypertension, hypertensive crisis, hypertensive urgency, isolated systolic and diastolic hypertension, pregnancy-related hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, angiopathy, diabetic nephropathy, diabetic retinopathy, chronic heart failure, cardiomyopathy, diabetic cardiomyopathy, glomerulosclerosis, coarctation of the aorta, aortic aneurysm, and ventricular fibrosis.


SEQUENCE LISTING (19 sequences)

SEQ ID NO: 1 - RNA, 19 nt, synthetic construct
  source 1..19: mol_type = other RNA
catccacaatgagagtacc

SEQ ID NO: 2 - RNA, 21 nt, synthetic construct
  source 1..21: mol_type = other RNA
ggtactctcattgtggatgac

SEQ ID NO: 3 - RNA, 19 nt, synthetic construct
  source 1..19: mol_type = other RNA
tcaactggatgaagaaact

SEQ ID NO: 4 - RNA, 21 nt, synthetic construct
  source 1..21: mol_type = other RNA
agtttcttcatccagttgagg

SEQ ID NO: 5 - RNA, 19 nt, synthetic construct
  source 1..19: mol_type = other RNA
cctgtttgctgtgtatgat

SEQ ID NO: 6 - RNA, 21 nt, synthetic construct
  source 1..21: mol_type = other RNA
atcatacacagcaaacaggaa

SEQ ID NO: 7 - RNA, 19 nt, synthetic construct
  source 1..19: mol_type = other RNA
caagttgagaacaaaaatt

SEQ ID NO: 8 - RNA, 21 nt, synthetic construct
  source 1..21: mol_type = other RNA
aatttttgttctcaacttgaa

SEQ ID NO: 9 - RNA, 19 nt, synthetic construct
  source 1..19: mol_type = other RNA
gttttaaaattaaagtata

SEQ ID NO: 10 - RNA, 21 nt, synthetic construct
  source 1..21: mol_type = other RNA
tatactttaattttaaaaccc

SEQ ID NO: 11 - RNA, 19 nt, synthetic construct
  source 1..19: mol_type = other RNA
aagtatacatttttgcatt

SEQ ID NO: 12 - RNA, 21 nt, synthetic construct
  source 1..21: mol_type = other RNA
aatgcaaaaatgtatacttta

SEQ ID NO: 13 - DNA, 19 bp, synthetic construct
  source 1..19: mol_type = other DNA
gtatgtacacccggtcacc

SEQ ID NO: 14 - DNA, 21 bp, synthetic construct
  source 1..21: mol_type = other DNA
gttgttctgggtactacagca

SEQ ID NO: 15 - DNA, 21 bp, synthetic construct
  source 1..21: mol_type = other DNA
catcctctgcctcctggcctg

SEQ ID NO: 16 - DNA, 19 bp, synthetic construct
  source 1..19: mol_type = other DNA
aagaaggtggtgaagcagg

SEQ ID NO: 17 - DNA, 18 bp, synthetic construct
  source 1..18: mol_type = other DNA
caaagtggtcgttgaggg

SEQ ID NO: 18 - DNA, 18 bp, synthetic construct
  source 1..18: mol_type = other DNA
caacagcgacacccactc

SEQ ID NO: 19 - DNA, 2116 bp, synthetic construct
  source 1..2116: mol_type = other DNA
gaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaa